(19) United States
(12) Patent Application Publication
(10) Pub. No.: US 2006/0160220 A1
(43) Pub. Date: Jul. 20, 2006

(54) Retroviral vectors with introns

(75) Inventors: Robert Bremel, Hillpoint, WI (US); Jerome S. Harms, Madison, WI (US)

Correspondence Address: Medlen & Carroll, LLP, Suite 350, 101 Howard Street, San Francisco, CA, 94105, US

(73) Assignee: ioGenetics, Madison, WI (US)

(21) Appl. No.: 11/273,388

(22) Filed: Nov. 14, 2005

Related U.S. Application Data
(60) Provisional application No. 60/627,693, filed on Nov. 12, 2004.

(51) Int. Cl. C12N 15/867

(52) U.S. Cl. 435/456

(57) ABSTRACT

The present invention relates to improved retroviral vectors. In particular, the present invention relates to retroviral vectors that retain introns in genes of interest during vector production. The present invention further provides host cells and animals comprising gene delivered by the vectors. The present invention additionally provides methods of using such retroviral vectors, host cells and animals in research, diagnostic and therapeutic applications.

This application claims priority to provisional patent application serial number 60/627,693, filed Nov. 12, 2004, which is herein incorporated by reference in its entirety.

This application was supported in part by SBIR grant No. R44CA88752. The government may have certain rights in the invention;

### FIELD OF THE INVENTION

The present invention relates to improved retroviral vectors. In particular, the present invention relates to retroviral vectors that retain introns in genes of interest during vector production. The present invention further provides host cells and animals comprising genes delivered by the vectors and thus retaining introns. The present invention additionally provides methods of using such retroviral vectors, host cells and animals in research, diagnostic and therapeutic applications.

### BACKGROUND OF THE INVENTION

Retrovectors have been used for gene transfer in a variety of experimental, medical and industrial settings including the creation of protein production cell lines for pharmaceutical and other recombinant protein manufacturing purposes and for the creation of transgenic animals to produce proteins of commercial interest or to confer disease resistance traits. Retrovectors are one of the principal tools used for the delivery of genes in gene therapy to treat deficiency diseases and otherwise deliver exogenous genes in vivo. Retrovectors are tools widely used in the research laboratory to elucidate the function of specific genes and there will be a continuing need for such research tools as functional genomics continues to develop as a field of inquiry underpinning medicine and drug development.

Retrovectors provide an effective means of gene transfer in these situations because they bring about stable integration in the genome of the host cells of proviruses containing the genes of interest. Additional efficacy and efficiency has been provided through the use of retrovectors that are pseudotyped with VSVG to create pantropism and stabilize the retrovectors to allow preparation of high titer concentrations of the vectors (Yee et al., PNAS 91:9564 [1994]). This enhancement has been utilized widely in production of transgenics, in research and in the practice of gene therapy.

Retrovector particles are assembled by export of the genes of interest from the nucleus of packaging cells encoded in viral genomic RNA and assembed into retrovector particles along with protein products of gag, pol and env. In the absence of virally coded mechanisms to protect the gene of interest from splicing, the RNA for the gene of interest carried out of the packaging cells in mature retrovector particles is spliced, removing any introns that may be present in the gene. When such genes are introduced into target host cells by the retrovector the expression of a gene may fail or be reduced or otherwise modified in the absence of introns. This is a limitation of currently available retrovector systems.

The utility of retrovectors in all of their applications would be enhanced by the availability of retrovectors that retain the presence and function of introns in the genes of interest.

### SUMMARY OF THE INVENTION

The present invention relates to improved retroviral vectors. In particular, the present invention relates to retroviral vectors that retain introns in genes of interest during vector production. The present invention further provides host cells and animals comprising genes delivered by the vectors and thus retaining introns. The present invention additionally provides methods of using such retroviral vectors, host cells and animals in research, diagnostic and therapeutic applications.

Accordingly, in some embodiments, the present invention provides a system, comprising: a retroviral vector comprising a promoter operably linked to a nucleic acid encoding an exogenous gene and a nucleic acid encoding an RNA export protein response element; and a packaging cell line expressing an RNA export protein. In some embodiments, the RNA export protein response element is a Rex RNA response element (RxRE) (e.g., a bovine leukemia virus RxRE or a human T-cell leukemia RxRe). In some embodiments, the bovine leukemia virus RxRE is at least 90% identical to SEQ ID NO:5. In other embodiments, the bovine leukemia virus RxRE has the nucleic acid sequence of SEQ ID NO:5. In some embodiments, the human T Cell leukemia virus RxRE is at least 90% identical to SEQ ID NO:4. In other embodiments, the human T Cell leukemia virus RxRE has the nucleic acid sequence of SEQ ID NO:4. In some embodiments, the RNA export protein response element is a human immunodeficiency virus RRE. In some embodiments, the human immunodeficiency virus RRE is at least 90% identical to SEQ ID NO: 6. In other embodiments, the human immunodeficiency virus RRE has the nucleic acid sequence of SEQ ID NO: 6. In further embodiments, the RNA export protein is a bovine leukemia virus Rex or a human T-cell leukemia virus Rex. In some embodiments, the bovine leukemia virus Rex is at least 90% identical to SEQ ID NO:2. In other embodiments, the bovine leukemia virus Rex has the nucleic acid sequence of SEQ ID NO:2. In some embodiments, the human T-cell leukemia virus Rex is at least 90% identical to SEQ ID NO:7. In other embodiments, the human T-cell leukemia virus Rex has the nucleic acid sequence of SEQ ID NO:7. In still other embodiments, the nuclear export protein is human immunodeficiency virus Rev. In some embodiments, the human immunodeficiency virus Rev is at least 90% identical to SEQ ID NO:3. In other embodiments, the human immunodeficiency virus Rev has the nucleic acid sequence of SEQ ID NO:3. In certain embodiments, the RNA export protein is present on a second vector. In some embodiments, the second vector is a lentiviral vector or MLV vector. In certain embodiments, the second vector is an inducible expression vector (e.g., comprises a tet responsive element). In some embodiments, the RNA export protein is present as a transgene. In certain embodiments, the retroviral vector further comprises an RNA stabilizing element (e.g., a WPRE).

The present invention further provides a method, comprising: providing a retroviral vector comprising a promoter operably linked to a nucleic acid encoding an exogenous gene and a nucleic acid encoding an RNA export protein response element; and a packaging cell line expressing an RNA export protein; and introducing the retroviral vector into the packaging cell line under conditions such that the retroviral vector is packaged without introns being spliced from the exogenous gene. In some embodiments, the RNA export protein response element is a Rex RNA response element (RxRE) (e.g., a bovine leukemia virus RxRE or a human T-cell leukemia RxRe). In some embodiments, the bovine leukemia virus RxRE is at least 90% identical to SEQ ID NO:5. In other embodiments, the bovine leukemia virus RxRE has the nucleic acid sequence of SEQ ID NO:5. In some embodiments, the human T Cell leukemia virus RxRE is at least 90% identical to SEQ ID NO:4. In other embodiments, the human T Cell leukemia virus RxRE has the nucleic acid sequence of SEQ ID NO:4. In some embodiments, the RNA export protein response element is a human immunodeficiency virus RRE. In some embodiments, the human immunodeficiency virus RRE is at least 90% identical to SEQ ID NO: 6. In other embodiments, the human immunodeficiency virus RRE has the nucleic acid sequence of SEQ ID NO: 6. In further embodiments, the RNA export protein is a bovine leukemia virus Rex or a human T-cell leukemia virus Rex. In some embodiments, the bovine leukemia virus Rex is at least 90% identical to SEQ ID NO:2. In other embodiments, the bovine leukemia virus Rex has the nucleic acid sequence of SEQ ID NO:2. In some embodiments, the human T-cell leukemia virus Rex is at least 90% identical to SEQ ID NO:7. In other embodiments, the human T-cell leukemia virus Rex has the nucleic acid sequence of SEQ ID NO:7. In still other embodiments, the nuclear export protein is human immunodeficiency virus Rev. In some embodiments, the human immunodeficiency virus Rev is at least 90% identical to SEQ ID NO:3. In other embodiments, the human immunodeficiency virus Rev has the nucleic acid sequence of SEQ ID NO:3. In certain embodiments, the RNA export protein is present on a second vector. In some embodiments, the second vector is a lentiviral vector or MLV vector. In certain embodiments, the second vector is an inducible expression vector (e.g., comprises a tet responsive element). In some embodiments, the RNA export protein is present as a transgene. In certain embodiments, the retroviral vector further comprises an RNA stabilizing element (e.g., a WPRE).

The present invention further provides a retroviral vector comprising a promoter operably linked to a nucleic acid encoding an exogenous gene and a nucleic acid encoding an RNA export protein response element. In some embodiments, the RNA export protein response element is a Rex RNA response element (RxRE) (e.g., a bovine leukemia virus RxRE or a human T-cell leukemia RxRe). In some embodiments, the bovine leukemia virus RxRE is at least 90% identical to SEQ ID NO:5. In other embodiments, the bovine leukemia virus RxRE has the nucleic acid sequence of SEQ ID NO:5. In some embodiments, the human T Cell leukemia virus RxRE is at least 90% identical to SEQ ID NO:4. In other embodiments, the human T Cell leukemia virus RxRE has the nucleic acid sequence of SEQ ID NO:4. In some embodiments, the RNA export protein response element is a human immunodeficiency virus RRE. In some embodiments, the human immunodeficiency virus RRE is at least 90% identical to SEQ ID NO: 6. In other embodiments, the human immunodeficiency virus RRE has the nucleic acid sequence of SEQ ID NO: 6. In some embodiments, the retroviral vector further comprises an RNA stabilizing element (e.g., a WPRE). The present invention further provides a host cell comprising the retroviral vector (e.g., a stem cell or a protein production cell). The present invention further provides a transgenic animal or plant comprising the vector. The present invention also provides an animal comprising the host cell (e.g., a human or a non-human mammal).

In yet other embodiments, the present invention provides a host cell comprising a genome, wherein the genome comprises a transgene delivered by a retroviral vector, and wherein the transgene comprises introns. In some embodiments, the host cell is a packaging cell line, a protein production cell, or a stem cell. In other embodiments, the host cell is in a transgenic animal or plant.

In still further embodiments, the present invention provides a retroviral packaging cell line comprising an exogenous RNA export protein gene. In some embodiments, the exogenous RNA export protein gene is a gene encoding a bovine leukemia virus Rex or a human T-cell leukemia virus Rex. In some embodiments, the bovine leukemia virus Rex is at least 90% identical to SEQ ID NO:2. In other embodiments, the bovine leukemia virus Rex has the nucleic acid sequence of SEQ ID NO:2. In some embodiments, the human T-cell leukemia virus Rex is at least 90% identical to SEQ ID NO:7. In other embodiments, the human T-cell leukemia virus Rex has the nucleic acid sequence of SEQ ID NO:7. In still other embodiments, the nuclear export protein is human immunodeficiency virus Rev. In some embodiments, the human immunodeficiency virus Rev is at least 90% identical to SEQ ID NO:3. In other embodiments, the human immunodeficiency virus Rev has the nucleic acid sequence of SEQ ID NO:3. In some embodiments, the cell line further expresses at least one of the genes encoding gag, pol, and env of a retrovirus. In certain embodiments, the gene encoding the nuclear export protein is stably integrated. In other embodiments, the gene encoding the nuclear export protein is transiently introduced into the packaging cell. In some embodiments, at least one of the genes encoding gag, pol, and env of a retrovirus and the gene encoding the nuclear export protein are integrated at different locations in the genome of the packaging cell line.

The present invention further provides a method, comprising providing a cell suspected of harboring a viral infection; and a retroviral vector comprising a promoter operably linked to a nucleic acid encoding an exogenous gene and a nucleic acid encoding an RNA export protein response element, wherein the retroviral vector further comprises a reporter gene; and transfecting the cell with the retroviral vector under conditions such that the reporter gene is expressed in the presence but not in the absence of the viral infection. In some embodiments, the viral infection is infection with human immunodeficiency virus and the RNA export protein response element is human immunodeficiency RRE. In other embodiments, the viral infection is infection with bovine leukemia virus and the RNA export protein response element is bovine leukemia virus RxRE. In still other embodiments, the viral infection is infection with human T cell leukemia virus and the RNA export protein response element is human T cell leukemia virus RxRE. In some embodiments, the cells are derived from an animal (e.g., a human).

### DESCRIPTION OF THE FIGURES

FIG. 1 shows a map of the RxRe vector used in some embodiments of the present invention.

FIG. 2 shows RxRe reporter activity in stably transfected cell lines. FIG. 2A shows activity in a non-BLV expressing cell line and FIG. 2B shows activity in a BLV expressing cell line.

FIG. 3 shows RxRe reporter activity in the presence of TD-Rex mutants.

FIG. 4 shows the nucleic acid sequence of pLNCXBXREG (SEQ ID NO:1).

FIG. 5 provides the nucleic acid sequence for BLV Rex (SEQ ID NO:2).

FIG. 6 provides the nucleic acid sequence for HIV Rev (SEQ ID NO:3).

FIG. 7 provides the nucleic acid sequence for HTLV RxRe (SEQ ID NO:4).

FIG. 8 provides the nucleic acid sequence for BLV RxRE (SEQ ID NO:5).

FIG. 9 provides the nucleic acid sequence for HIV RRE (SEQ ID NO:6).

FIG. 10 provides the nucleic acid sequence for HTLV Rex (SEQ ID NO:7).

FIG. 11 shows a map of pLNCXBXRE/SEAP.

FIG. 12 shows alkaline phosphatase assay for detection of SEAP.

FIG. 13 shows the nucleic acid sequence of pLNCXBXRE/SEAP.

### DEFINITIONS

To facilitate an understanding of the present invention, a number of terms and phrases are defined below:

As used herein, the term “post transcriptional regulatory element (PRE)” refers to RNA stabilizing elements derived from RNA viruses, in particular hepadna viruses. In some embodiments, PREs include, but are not limited to, the WPRE of woodchuck hepatitis virus, and the post transcriptional regulatory element of Hepatitis B virus. PREs are also referred to as RNA Export Stabilizing Elements.

As used herein, the term “RNA export protein” refers to a protein that regulates the export of RNA from the host cell nucleus. In some embodiments, viral RNA export proteins include, but are not limited to, the Rev proteins of lentiviruses and the Rex proteins of the HTLV-BLV group of complex retroviruses. Each binds to an RNA export protein response element and facilitates the transport of unspliced and incompletely spliced RNAs to the cytoplasm.

As used herein, the term “RNA export protein response element” refers to a region of RNA in the 3′ and 5′ LTRs of viral nucleic acids that RNA export proteins bind to in order to regulate export of RNA from the nucleus. Examples of RNA export protein response elements include, but are not limited to, BLV and HTLV Rex response elements (“RxRE”) and HIV Rev response elements (“RRE”).

As used herein, the term “host cell” refers to any eukaryotic cell (e.g., mammalian cells, avian cells, amphibian cells, plant cells, fish cells, and insect cells), whether located in vitro or in vivo (e.g., in a transgenic organism).

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “vector” refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors.

As used herein, the term “integrating vector” refers to a vector whose integration or insertion into a nucleic acid (e.g., a chromosome) is accomplished via an integrase. Examples of “integrating vectors” include, but are not limited to, retroviral vectors, transposons, and adeno associated virus vectors.

As used herein, the term “integrated” refers to a vector that is stably inserted into the genome (i.e., into a chromosome) of a host cell.

As used herein, the term “genome” refers to the genetic material (e.g., chromosomes) of an organism or a host cell.

The term “nucleotide sequence of interest” refers to any nucleotide sequence (e.g., RNA or DNA), the manipulation of which may be deemed desirable for any reason (e.g. treat disease, confer improved qualities, etc.), by one of ordinary skill in the art. Such nucleotide sequences include, but are not limited to, coding sequences, or portions thereof, of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

The term “gene” refers to a nucleic acid (e.g., DNA or RNA) sequence that comprises coding sequences necessary for the production of a polypeptide or precursor (e.g., proinsulin). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “exogenous gene” refers to a gene that is not naturally present in a host organism or cell, or is artificially introduced into a host organism or cell.

As used herein, the term “gene of interest” refers to any gene for which the manipulation may be deemed desirable for any reason (e.g., treat disease, confer improved qualities, etc.), by one of ordinary skill in the art.

As used herein, term “BLV rex gene” (or B rex) refers to the full-length BLV rex nucleotide sequence (e.g., contained in SEQ ID NO: 2). However, it is also intended that the term encompass fragments of the B rex sequence, as well as other domains within the full-length B rex nucleotide sequence. Furthermore, the terms “B rex nucleotide sequence” or “B rex polynucleotide sequence” encompasses DNA, cDNA, and RNA (e.g., mRNA) sequences. Similarly, the term “HTLV rex gene” (or H rex) refers to the full-length HTLV rex nucleotide sequence. However, it is also intended that the term encompass fragments of the H rex sequence, as well as other domains within the full-length H rex nucleotide sequence. Furthermore, the terms “H rex nucleotide sequence” or “H rex polynucleotide sequence” encompasses DNA, cDNA, and RNA (e.g., mRNA) sequences.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decreases production. Molecules (e.g., transcription factors) that are involved in up-regulation or down-regulation are often called “activators” and “repressors,” respectively.

As used herein, the term “protein of interest” refers to a protein encoded by a nucleic acid of interest.

As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain translated from the mRNA. The DNA or RNA sequence thus codes for the amino acid sequence.

As used herein, the term “native” (or wild type) when used in reference to a protein, refers to proteins encoded by partially homologous nucleic acids so that the amino acid sequence of the proteins varies. As used herein, the term “variant” encompasses proteins encoded by homologous genes having both conservative and nonconservative amino acid substitutions that do not result in a change in protein function, as well as proteins encoded by homologous genes having amino acid substitutions that cause decreased (e.g., null mutations) protein function or increased protein function.

As used herein the term “retroviral processing protein” refers to a protein or polypeptide that functions to promote retroviral replication. Examples of retroviral processing proteins include, but are not limited to, BRex, HRex, and Rev proteins or functional polypeptides.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology” and “percent identity” when used in relation to nucleic acids refer to a degree of complementarity. There may be partial homology (i.e., partial identity) or complete homology (i.e., complete identity). A partially complementary sequence is one that at least partially inhibits a completely complementary sequence from hybridizing to a target nucleic acid sequence and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe (i.e., an oligonucleotide which is capable of hybridizing to another oligonucleotide of interest) will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.” As used herein, the term “Tₘ” is used in reference to the “melting temperature” of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization[1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less. “High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42 C when a probe of about 500 nucleotides in length is employed. “Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42 C when a probe of about 500 nucleotides in length is employed. “Low stringency conditions” comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42 C when a probe of about 500 nucleotides in length is employed.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “selectable marker” refers to a gene that encodes an enzymatic activity that confers the ability to grow in medium lacking what would otherwise be an essential nutrient (e.g., the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include, but are not limited to, the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk - cell lines, the CAD gene which is used in conjunction with CAD-deficient cells and the mammalian hypoxanthine-guanine phosphoribosyl transferase (hprt) gene which is used in conjunction with hprt—cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp.16.9-16.15.

As used herein, the term “reporter gene” refers to a gene encoding a protein that may be assayed. Examples of reporter genes include, but are not limited to, luciferase (See, e.g., deWet et al., Mol. Cell. Biol. 7:725 [1987] and U.S. Pat Nos.,6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

As used herein, the term “regulatory element” refers to a genetic element which controls some aspect of the expression of nucleic acid sequences. For example, a promoter is a regulatory element that facilitates the initiation of transcription of an operably linked coding region. Other regulatory elements are splicing signals, polyadenylation signals, termination signals, RNA export elements, internal ribosome entry sites, etc. (defined infra).

Transcriptional control signals in eukaryotes comprise “promoter” and “enhancer” elements. Promoters and enhancers consist of short arrays of DNA sequences that interact specifically with cellular proteins involved in transcription (Maniatis et al., Science 236:1237 [1987]). Promoter and enhancer elements have been isolated from a variety of eukaryotic sources including genes in yeast, insect and mammalian cells, and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see, Voss et al., Trends Biochem. Sci., 11:287 [1986]; and Maniatis et al., supra). For example, the SV40 early gene enhancer is very active in a wide variety of cell types from many mammalian species and has been widely used for the expression of proteins in mammalian cells (Dijkema et al., EMBO J. 4:761 [1985]). Two other examples of promoter/enhancer elements active in a broad range of mammalian cell types are those from the human elongation factor 1 gene (Uetsuki et al., J. Biol. Chem., 264:5791 [1989]; Kim et al., Gene 91:217 [1990]; and Mizushima and Nagata, Nuc. Acids. Res., 18:5322 [1990]) and the long terminal repeats of the Rous sarcoma virus (Gorman et al., Proc. Natl. Acad. Sci. USA 79:6777 [1982]) and the human cytomegalovirus (Boshart et al., Cell 41:521 [1985]).

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques such as cloning and recombination) such that transcription of that gene is directed by the linked enhancer/promoter.

Regulatory elements may be tissue specific or cell specific. The term “tissue specific” as it applies to a regulatory element refers to a regulatory element that is capable of directing selective expression of a nucleotide sequence of interest to a specific type of tissue (e.g., liver) in the relative absence of expression of the same nucleotide sequence of interest in a different type of tissue (e.g., lung).

Tissue specificity of a regulatory element may be evaluated by, for example, operably linking a reporter gene to a promoter sequence (which is not tissue-specific) and to the regulatory element to generate a reporter construct, introducing the reporter construct into the genome of an animal such that the reporter construct is integrated into every tissue of the resulting transgenic animal, and detecting the expression of the reporter gene (e.g., detecting mRNA, protein, or the activity of a protein encoded by the reporter gene) in different tissues of the transgenic animal. The detection of a greater level of expression of the reporter gene in one or more tissues relative to the level of expression of the reporter gene in other tissues shows that the regulatory element is “specific” for the tissues in which greater levels of expression are detected. Thus, the term “tissue-specific” (e.g., liver-specific) as used herein is a relative term that does not require absolute specificity of expression. In other words, the term “tissue-specific” does not require that one tissue have extremely high levels of expression and another tissue have no expression. It is sufficient that expression is greater in one tissue than another. By contrast, “strict” or “absolute” tissue-specific expression is meant to indicate expression in a single tissue type (e.g., liver) with no detectable expression in other tissues.

The term “cell type specific” as applied to a regulatory element refers to a regulatory element which is capable of directing selective expression of a nucleotide sequence of interest in a specific type of cell in the relative absence of expression of the same nucleotide sequence of interest in a different type of cell within the same tissue (e.g., cells infected with retrovirus, and more particularly, cells infected with BLV or HTLV). The term “cell type specific” when applied to a regulatory element also means a regulatory element capable of promoting selective expression of a nucleotide sequence of interest in a region within a single tissue.

Cell type specificity of a regulatory element may be assessed using methods well known in the art (e.g., immunohistochemical staining and/or Northern blot analysis). Briefly, for immunohistochemical staining, tissue sections are embedded in paraffin, and paraffin sections are reacted with a primary antibody specific for the polypeptide product encoded by the nucleotide sequence of interest whose expression is regulated by the regulatory element. A labeled (e.g., peroxidase conjugated) secondary antibody specific for the primary antibody is allowed to bind to the sectioned tissue and specific binding detected (e.g., with avidin/biotin) by microscopy. Briefly, for Northern blot analysis, RNA is isolated from cells and electrophoresed on agarose gels to fractionate the RNA according to size followed by transfer of the RNA from the gel to a solid support (e.g., nitrocellulose or a nylon membrane). The immobilized RNA is then probed with a labeled oligo-deoxyribonucleotide probe or DNA probe to detect RNA species complementary to the probe used. Northern blots are a standard tool of molecular biologists.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, etc.). In contrast, a “regulatable” promoter is one which is capable of directing a level of transcription of an operably linked nucleic acid sequence in the presence of a stimulus (e.g., heat shock, chemicals, etc.) which is different from the level of transcription of the operably linked nucleic acid sequence in the absence of the stimulus.

The presence of “splicing signals” on an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York [1989], pp. 16.7-16.8). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly A site” or “poly A sequence” as used herein denotes a DNA sequence that directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one that is isolated from one gene and placed 3′ of another gene. A commonly used heterologous poly A signal is the SV40 poly A signal. The SV40 poly A signal is contained on a 237 bp BamHI/Bcll restriction fragment and directs both termination and polyadenylation (Sambrook, supra, at 16.6-16.7).

Eukaryotic expression vectors may also contain “viral replicons ” or “viral origins of replication.” Viral replicons are viral DNA sequences that allow for the extrachromosomal replication of a vector in a host cell expressing the appropriate replication factors. Vectors that contain either the SV40 or polyoma virus origin of replication replicate to high “copy number” (up to 10⁴ copies/cell) in cells that express the appropriate viral T antigen. Vectors that contain the replicons from bovine papillomavirus or Epstein-Barr virus replicate extrachromosomally at “low copy number” (˜100 copies/cell). However, it is not intended that expression vectors be limited to any particular viral origin of replication.

As used herein, the term “long terminal repeat” or “LTR” refers to transcriptional control elements located in or isolated from the U3 region 5′ and 3′ of a retroviral genome. As is known in the art, long terminal repeats may be used as control elements in retroviral vectors, or isolated from the retroviral genome and used to control expression from other types of vectors.

As used herein, the terms “RNA export element” or “Pre-mRNA Processing Enhancer (PPE)” refer to 3′ and 5′ cis-acting post-transcriptional regulatory elements that enhance export of RNA from the nucleus. “PPE” elements include, but are not limited to Mertz sequences (described in U.S. Pat. Nos. 5,914,267 and 5,686,120, all of which is incorporated herein by reference) and woodchuck mRNA processing enhancer (WPRE; WO99/143 10, incorporated herein by reference).

As used herein, the term “polycistronic” refers to an mRNA encoding more than polypeptide chain (See, e.g., WO 93/03143, WO 88/05486, and European Pat. No. 117058, all of which is incorporated herein by reference). Likewise, the term “arranged in polycistronic sequence” refers to the arrangement of genes encoding two different polypeptide chains in a single mRNA.

As used herein, the term “internal ribosome entry site” or “IRES” refers to a sequence located between polycistronic genes that permits the production of the expression product originating from the second gene by internal initiation of the translation of the dicistronic mRNA. Examples of internal ribosome entry sites include, but are not limited to, those derived from foot and mouth disease virus (FDV), encephalomyocarditis virus, poliovirus and RDV (Scheper et al., Biochem. 76: 801-809 [1994]; Meyer et al., J. Virol. 69: 2819-2824 [1995]; Jang et al., 1988, J. Virol. 62: 2636-2643 [1998]; Haller et al., J. Virol. 66: 5075-5086 [1995]). Vectors incorporating IRES's may be assembled as is known in the art. For example, a retroviral vector containing a polycistronic sequence may contain the following elements in operable association: nucleotide polylinker, gene of interest, an internal ribosome entry site and a mammalian selectable marker or another gene of interest. The polycistronic cassette is situated within the retroviral vector between the 5′ LTR and the 3′ LTR at a position such that transcription from the 5′ LTR promoter transcribes the polycistronic message cassette. The transcription of the polycistronic message cassette may also be driven by an internal promoter (e.g., cytomegalovirus promoter) or an inducible promoter, which may be preferable depending on the use. The polycistronic message cassette can further comprise a cDNA or genomic DNA (gDNA) sequence operatively associated within the polylinker. Any mammalian selectable marker can be utilized as the polycistronic message cassette mammalian selectable marker. Such mammalian selectable markers are well known to those of skill in the art and can include, but are not limited to, kanamycin/G418, hygromycin B or mycophenolic acid resistance markers.

As used herein, the terms “retrovirus” and “retrovector” are used interchangeably to refer to virus a with an RNA genome that is capable of entering a cell (i.e., the particle contains a membrane-associated protein such as an envelope protein which can bind to the host cell surface and facilitate entry of the viral particle into the cytoplasm of the host cell) and integrating the retroviral genome (as a double-stranded DNA provirus) into the genome of the host cell through the action of reverse transcriptase. The International Committee on Taxonomy of Virus defines 7 Genera of retrovirus: Alpharetrovirus (e.g., Avian leukosis virus); Betaretrovirus (e.g., Mouse mammary tumor virus); Gammaretrovirus (e.g., Murine leukemia virus); Deltaretrovirus (e.g., Bovine leukemia virus); Epsilonretrovirus (e.g., Walleye dermal sarcoma virus); Lentivirus (e.g., Human immunodeficiency virus 1); and Spumavirus (e.g., Chimpanzee foamy spumavirus).

As used herein, the term “retroviral vector” refers to a retrovirus that has been modified to express a gene of interest. Retroviral vectors can be used to transfer genes efficiently into host cells by exploiting the viral infectious process. Foreign or heterologous genes cloned (i.e., inserted using molecular biological techniques) into the retroviral genome can be delivered efficiently to host cells that are susceptible to infection by the retrovirus. Through well known genetic manipulations, the replicative capacity of the retroviral genome can be destroyed. The resulting replication-defective vectors can be used to introduce new genetic material to a cell but they are unable to replicate. A helper virus or packaging cell line can be used to permit vector particle assembly and egress from the cell. Such retroviral vectors comprise a replication-deficient retroviral genome containing a nucleic acid sequence encoding at least one gene of interest (i.e., a polycistronic nucleic acid sequence can encode more than one gene of interest), a 5′ retroviral long terminal repeat (5′ LTR); and a 3′ retroviral long terminal repeat (3′ LTR).

The term “pseudotyped retroviral vector” refers to a retroviral vector containing a heterologous membrane protein. The term “membrane-associated protein” refers to a protein (e.g., a viral envelope glycoprotein or the G proteins of viruses in the Rhabdoviridae family such as VSV, Piry, Chandipura and Mokola) that are associated with the membrane surrounding a viral particle; these membrane-associated proteins mediate the entry of the viral particle into the host cell. The membrane associated protein may bind to specific cell surface protein receptors, as is the case for retroviral envelope proteins or the membrane-associated protein may interact with a phospholipid component of the plasma membrane of the host cell, as is the case for the G proteins derived from members of the Rhabdoviridae family.

The term “heterologous membrane-associated protein” refers to a membrane-associated protein that is derived from a virus that is not a member of the same viral class, or family as that from which the nucleocapsid protein of the vector particle is derived. “Viral class or family” refers to the taxonomic rank of class or family, as assigned by the International Committee on Taxonomy of Viruses.

The term “Rhabdoviridae” refers to a family of enveloped RNA viruses that infect animals, including humans, and plants. The Rhabdoviridae family encompasses the genus Vesiculovirus, which includes vesicular stomatitis virus (VSV), Cocal virus, Piry virus, Chandipura virus, and Spring viremia of carp virus (sequences encoding the Spring viremia of carp virus are available under GenBank accession number U18101). The G proteins of viruses in the Vesiculovirus genera are virally-encoded integral membrane proteins that form externally projecting homotrimeric spike glycoproteins complexes that are required for receptor binding and membrane fusion. The G proteins of viruses in the Vesiculovirus genera have a covalently bound palmititic acid (C₁₆) moiety. The amino acid sequences of the G proteins from the Vesiculoviruses are fairly well conserved. For example, the Piry virus G protein share about 38% identity and about 55% similarity with the VSV G proteins (several strains of VSV are known, e.g., Indiana, N.J., Orsay, San Juan, etc., and their G proteins are highly homologous). The Chandipura virus G protein and the VSV G proteins share about 37% identity and 52% similarity. Given the high degree of conservation (amino acid sequence) and the related functional characteristics (e.g., binding of the virus to the host cell and fusion of membranes, including syncytia formation) of the G proteins of the Vesiculoviruses, the G proteins from non-VSV Vesiculoviruses may be used in place of the VSV G protein for the pseudotyping of viral particles. The G proteins of the Lyssa viruses (another genera within the Rhabdoviridae family) also share a fair degree of conservation with the VSV G proteins and function in a similar manner (e.g., mediate fusion of membranes) and therefore may be used in place of the VSV G protein for the pseudotyping of viral particles. The Lyssa viruses include the Mokola virus and the Rabies viruses (several strains of Rabies virus are known and their G proteins have been cloned and sequenced). The Mokola virus G protein shares stretches of homology (particularly over the extracellular and transmembrane domains) with the VSV G proteins which show about 31% identity and 48% similarity with the VSV G proteins. Preferred G proteins share at least 25% identity, preferably at least 30% identity and most preferably at least 35% identity with the VSV G proteins. The VSV G protein from which New Jersey strain (the sequence of this G protein is provided in GenBank accession numbers M27165 and M21557) is employed as the reference VSV G protein.

As used herein, the term “lentivirus vector” refers to retroviral vectors derived from the Lentiviridae family (e.g., human immunodeficiency virus, simian immunodeficiency virus, equine infectious anemia virus, and caprine arthritis-encephalitis virus) that are capable of integrating into non-dividing cells (See, e.g., U.S. Pat. Nos. 5,994,136 and 6,013,516, both of which are incorporated herein by reference).

The term “pseudotyped lentivirus vector” refers to lentivirus vector containing a heterologous membrane protein (e.g., a viral envelope glycoprotein or the G proteins of viruses in the Rhabdoviridae family such as VSV, Piry, Chandipura and Mokola).

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

### DETAILED DESCRIPTION OF THE INVENTION

The present invention relates to improved retroviral vectors. In particular, the present invention relates to retroviral vectors that retain introns in genes of interest during vector production. The present invention further provides host cells and animals comprising genes delivered by the vectors and thus retaining introns. The present invention additionally provides methods of using such retroviral vectors, host cells and animals in research, diagnostic and therapeutic applications.

I. Retroviral Gene Splicing and Export

For many decades, introns, those highly variable components of the genome that surround genes and gene components that are expressed as proteins, had been considered “junk DNA”. Recently the criticality of at least some introns has been recognized.

Experimenters who have made transgenic animals have demonstrated that expression can be enhanced by inclusion of introns with the transgenes (Cepko et al., Cell 37:1053 [1984]; Palmiter et al. PNAS 88:478 [1991]). There is evidence that microsatellites previously considered “junk” DNA may be an important source of quantative genetic variation (Ying et al., Gene 342:25 [2004]). Analysis of intron sizes in multiple species has shown they are non random and certain “minimal introns” are relatively conserved. Introns function in a wide variety of ways to influence many steps in RNA metabolism, including intron independent enhancement of transcription, interactions between splicing and other pre mRNA processing events, splicing and mRNA export, mRNA localization, translation, and decay of nonsense RNA (See e.g., Le Hir et al, Trends in Biochem. Sci 28:215 (2003)).

Prior to the present invention, retrovectors have long been known to eliminate introns (Shimotohno et al., Nature 299:265 [1982]; Cepko et al. supra; Kriegler et al., Cell 38:483 [1984]). Transcription of the cell-genome inserted provirus will initiate from one or more locations. Provided an active U3 element is present to provide enhancer activity, a full length viral RNA is produced that is initiated in the 5′ R region and terminating, with polyadenylation, in the 3′ R unit (a so-called SIN vector in which U3 is inactive can be made and this message will not be produced). When an internal promoter is used (e.g. CMV) a second mRNA is produced. This mRNA will initiate at the site engineered into the internal promoter and will likewise terminate, with polyadenylation, in the 3′ R unit.

Normally, an intron present in either of these type of transcripts is excised by the splicing machinery. The excision of introns is known to be intimately involved with export of mRNA into the cytoplasm for translation. Presence of an RNA structural element called RexRE in the RNA provides a site for interaction with the protein REX (so called because it provides an RNA export function). REX provides a second function besides an export activity in that it shields the mRNA from being spliced by the splicing apparatus. Thus, in cells that produce REX, full length messages produced from the incorporated provirus emanate from the nucleus with introns intact.

The structure and function of retroviruses and retrovectors have been described in detail. Retrovectors have been used in a broad variety of circumstances to deliver transgenes to eukaryotic cells in which a protein gene product of interest is desired to be produced. Examples include, but are not limited to, mammalian cell culture for protein production, the creation of transgenic animals expressing proteins of interest, transfer of genes to study gene function in cell culture experimental settings, and the delivery of genes for gene therapy. A shortcoming of retrovectors has been the splicing of introns from the genes of interest in the packaging cell lines used in the process of creating the RNA vectors (Shimotohno et al., supra). This has the effect detracting from the full function of the genes that are transferred to the recipient eukaryotic host cells. The transgene carried by the retrovector is a spliced version of the gene of interest from which the introns have been removed and which may not therefore reflect the full range of function or variability of the original gene of interest, once expressed in the recipient cell. Furthermore the splicing has “fixed” the transgene in one splice variant and eliminated the possibility of alternate splice variants being produced with different functions. The presence of introns and process of splicing in the host cell enhances the export of mRNA and hence protein expression by the host cell. Thus genes delivered by current retrovectors (without introns) have reduced protein expression and some fail to express the gene of interest (GOI) or gene transcription is modified in the host cell.

Eukaryotic gene expression depends on the synthesis, processing, export and translation of RNA from the nucleus. A number of pathways have been described for this process. The primary pathway from RNA export in eukaryotic cells involves splicing to generate mRNA. A number of Nuclear Export Factors (NXF) have been described which bind to polyadenylated RNAs and assist their passage to the cytoplasm (Izaurralde, EMBL Research Reports 2001 pp 1-5). Also essential to export of mRNAs are the “REF”s which are RNA binding proteins, a highly conserved family of proteins which form part of the exon exon junction complex deposited by the spliceosome. Export of intronless RNA or the splicing out of introns is an essential feature of RNA export by this pathway. The presence of introns enhances export of mRNA through the presence and/or increase of exon junction complexes, which bind proteins such as NXFs and REFs that enhance export.

Viruses adopt a number of means to avoid splicing of viral genomic RNA. The pathway for RNA nuclear export used by hepatitis B and woodchuck hepatitis virus encodes intronless messages but depends on RNA posttranscriptional regulatory elements (PREs) (also known as RNA export and stabilization elements or RESE) for expression (Donello et al., J. Virol 72:5085 [1996]; Fomerod et al., Cell 90:1051 [1997]). The PREs do not act through a virally encoded protein, rather the PREs are cis acting RNA elements that assist export of RNA (Fomerod et al., surpra). An example of this is the woodchuck hepatitis virus PRE (WPRE). The art knows the use of WPRE and related RNA stabilizing elements as components of retrovectors to enhance expression (Zufferey et al., J. Virol. 73:2886 [1999]).

Simple retroviruses use cellular RNA binding proteins to mediate export. Viral cis acting RNA elements known as the constitutive transport elements (CTE) selectively bind host encoded TAP protein and mediate export. This pathway is independent of CRM1, also called beta exportin l(Popa et al. Mol. Cell. Biol. 22:2057 [2002]).

Yet another distinct pathway of RNA export is found in the complex retroviruses which include lentiviruses (such as HIV, feline immunodeficiency virus and equine infectious anemia) in which virally coded proteins such as Rev (in HIV), and the complex oncoretroviruses (including Human T cell leukemia and bovine leukemia virus) in which similar virally coded proteins called Rex enable export of unspliced RNA (Coffin et al., Retroviruses. 1997. Cold Spring Harbor Laboratory Press, Plainview N.Y.). Rex functions to mediate the export and expression of intron-containing viral RNAs encoding the Gag, Pol, and Env proteins which are needed to generate new retroviral particles.

The BLV and HTLV-1 retroviral genomes both encode Rex proteins. These proteins are herein identified as BRex and HRex, respectively. Rex is a 27 kD phosphorylated gene product that is critical for virus replication. Rex is derived from the X3′ region of the genome and is encoded by the same doubly spliced mRNA as Tax. The rex gene encodes two proteins (27 Da and 21 kDa). The function of the smaller protein is unknown. The 27kDa Rex protein, unlike Tax, does not directly regulate transcription, but indirectly increases the expression of retroviral structural genes (i.e., gag and env) and enzymatic genes (i.e., pol) by increasing transport of unspliced or singly spliced viral mRNA out of the nucleus into the cytoplasm of the infected cell. Once these mRNA transcripts enter the cytoplasm, expression of the structural proteins Gag and Env is initiated while expression of the regulatory proteins is concomitantly suppressed (See e.g., Hidaka, M., et al., EMBO J. 7:519 [1988]) or modulated (See e.g., Malim, M. H., et al., Nature, 335:181 [1988]). A doubly spliced mRNA transcript codes for the Rex protein itself, so as the concentration of Rex increases, it indirectly inhibits its own translation. This has implications with respect to the latency aspect of the HTLV virus.

Nuclear export of retroviral mRNA molecules occurs by the direct binding of Rex in a sequence specific region called the Rex Response Element (RexRE) in the 3′ and 5′ LTRs of the molecule. The RexRE is a RNA stem-looped region that is highly stable and is present in all retroviral mRNA molecules of those complex retroviruses that have Rex. This means that another element is required in order to regulate expression and this element is called the cis-acting repressive sequence (CRS). When Rex binds the RexRE sequence it overcomes the inhibitory effect of the CRS. Since only unprocessed or singly spliced mRNA molecules contain both elements, only these elements are targeted for export to the cytoplasm and are consequently regulated by Rex activity. The RexRE is also known to have an activity apart from that of Rex, mainly RexRE aids in the 3′ cleavage and polyadenylation of all HTLV-1 viral transcripts.

The ability of Rex to regulate expression of the BLV and HTLV-1 gag and env genes requires at least three functionally distinct activities: 1) nuclear and nucleolar localization (i.e., the capacity to be transported from the cytoplasmic site of synthesis of all proteins to the nucleus and there to be concentrated in the nucleolar region); 2) specific recognition (directly or indirectly) of the Rex responsive element sequence in viral RNAs; and 3) Rex effector activity. The Rex protein of HTLV-1 belongs to a family of proteins that use arginine-rich motifs (ARMs) to recognize their RNA targets.

Human Immunodeficiency Virus Type 1 (HIV-1) encodes a protein homologous to Rex known as Rev. Rev protein is like the Rex in that it is required for the expression of viral structural proteins and thus production of competent viruses. In HIV-1, the selectivity of the induction noted above is due to an RNA target sequence required for Rev function termed Rev Response Element (RRE). RRE coincides with a large, 234 nucleotide RNA secondary structure present within the HIV-1 env gene.

The importance of Rex and Rev in the replication of complex retroviruses, respectively, is underscored by the fact that in spite of having different primary structures, Rex and Rev proteins are functionally related. For example, it is possible to substitute functional HTLV-1 Rex for defective Rev in the HIV-1 system, moreover, it has recently been found that HTLV-1 Rex and HIV-1 Rev can substitute for HIV-2 Rev (Rev2) and that HTLV-1 Rex can also substitute for the analogous HTLV-2 regulatory protein. (See e.g., Rimsky, L., et al., Nature, 335:738 [1988]). This complementation is sufficient to rescue rev-deficient HIV-1 provirus by providing functional Rex protein in trans. On the other hand, attempts to rescue a rex-deficient HTLV-1 provirus by addition of a functional Rev protein have been unsuccessful.

II. Retroviral System for Maintaining Introns

In some embodiments, the present invention provides retroviral systems for the expression or transfer of genes of interest containing introns. In some embodiments, the retroviral systems include viral RNA export proteins and their response elements as described above. Each of the components of the system is described in greater detail below.

A. Delivery of Rev or Rex

In some embodiments, the present invention provides retroviral expression and delivery systems that provide genes of interest with intact introns. In some embodiments, these systems comprise RNA export systems. The Rex or Rev components of these systems are provided to the cell separately from the corresponding RxRe or RRE response elements. In preferred embodiments, Rex proteins are paired with RxRE response elements and Rev proteins are paired with RRE elements. However, it is contemplated that Rex proteins may also be paired with RRE elements and Rev paired with RxRE elements.

In some embodiments, genes encoding for Rev or Rex proteins are included as transgenes in packaging cell lines. The genes encoding Rex or Rev may be introduced by any method known in the art, including, but not limited to transformation with a plasmid, retrovector transduction, lipofection, calcium phosphate precipitation, microinjection, electroporation, etc. In other embodiments, a construct (e.g., a plasmid) encoding Rex or Rev is transiently introduced into the packaging cells. In preferred embodiments, vectors used to create packaging cell lines incorporating Rex deliver it under the control of a different packaging signal from that which will be used for the gene of interest in order to prevent Rex from being packaged into the retrovector used to target the eventual host cell.

In some embodiments, the following exemplary vectors are used to create Rex or Rev containing packaging cell lines or to deliver Rex or Rev to a host cell other than a packaging cell line. In certain embodiments, vectors for conditional intron excision are utilized. In preferred embodiments, the Rex or Rev delivery vector is a different vector type from the vector containing the response element and gene of interest. This is preferred in order to avoid packaging of the Rex or Rev protein in the final viral particle. For example, in embodiments, where the response element and gene of interest are contained on a MLV vector, a lentiviral vector is utilized for the delivery of Rex or Rev. Conversely, if the response element and gene of interest are contained on a lentiviral vector, a MLV vector is utilized for the delivery of Rex or Rev. Abbreviations used in the exemplary vectors described below are defined in Table 1 below.

Examples of a REX retrovector:

1. lenti-LTR-Ψ-Kozα-πKozGOI(REX)-γ-lenti-LTR

2. LTR-Ψ-Kozα-πrKozGOI(REX)-γ-LTR

In other embodiments, a MLV vector is utilized:

3. πKozGOI(REX)-γ-SV40PolyadenylationSite

In some embodiments, this construct is transiently transfected into the packaging cells as are VSV-G and the plasmids containing the gene of interest, rather than used in a prior step to create a Rex modified base packaging line. In some embodiments, retrovectors for conditional expression of REX with the “tet” system, which could be either tet-on or tet off are utilized:

4. lenti-LTR-Ψ-Kozα-π“tet”KozGOI(REX)-γ-lenti-LTR

5. LTR-Ψ-Koza-π“tet”KozGOI(REX)-γ-LTR

The present invention is not limited to a particular Rex or Rev sequence. In some embodiments, Rex is derived from Bovine leukemia virus (BLV). In certain embodiments, the BLV Rex protein described by SEQ ID NO:2 is utilized. In other embodiments, HTLV Rex is utilized. In some embodiments, HIV Rev is utilized (e.g., the HIV Rev described by SEQ ID NO:3).

In general, for safety reasons, many recombinant retroviral vectors lack functional copies of the genes that are essential for viral replication (these essential genes are either deleted or disabled); therefore, the resulting virus is said to be replication defective. Packaging cell lines provide proteins required in trans for the packaging of the viral genomic RNA into viral particles having the desired host range (i.e., the viral-encoded gag, pol and env proteins). The host range is controlled, in part, by the type of envelope gene product expressed on the surface of the viral particle. Packaging cell lines may express ecotrophic, amphotropic or xenotropic envelope gene products. Alternatively, the packaging cell line may lack sequences encoding a viral envelope (env) protein. In this case the packaging cell line will package the viral genome into particles that lack a membrane-associated protein (e.g., an env protein). In order to produce viral particles containing a membrane associated protein that will permit entry of the virus into a cell, the packaging cell line containing the retroviral sequences is transfected with sequences encoding a membrane-associated protein (e.g., the G protein of vesicular stomatitis virus (VSV)). The transfected packaging cell will then produce viral particles that contain the membrane-associated protein expressed by the transfected packaging cell line; these viral particles that contain viral genomic RNA derived from one virus encapsidated by the envelope proteins of another virus are said to be pseudotyped virus particles.

Thus, it is contemplated that the packaging cells of the present invention stably or transiently express gag, pol , and env proteins for a particular retroviral particle as well as Rex or Rev. These genes may be expressed in single genetic constructs, or preferably, are present in the host cells on different vectors or are integrated at different locations within the packaging cell genome. Rex and Rev constructs can be transiently or stably introduced into any number of packaging cell lines, including, but not limited to 293 gp, 293T, PA317, PT67, PG 13, ΨCRIP, ΨCRE (See Coffin, supra for additional packaging cell lines).

B. Retroviral Constructs

In some embodiments, the present invention provides retroviral constructs comprising a gene of interest and a Rev (RRE) or Rex (RxRe) response element. In some embodiments, the vectors comprise additional elements useful in the expression or delivery of a gene of interest containing introns.

The retroviral vectors of the present invention can be further modified to include additional regulatory sequences. As described below, the retroviral vectors of the present invention include the following elements in operable association: a) a 5′ LTR; b) a packaging signal; c) a 3′ LTR and d) a nucleic acid encoding a protein of interest located between the 5′ and 3′ LTRs. In some embodiments of the present invention, the nucleic acid of interest may be arranged in opposite orientation to the 5′ LTR when transcription from an internal promoter is desired. Suitable internal promoters include, but are not limited to, the alpha-lactalbumin promoter, the CMV promoter (human or ape), and the thymidine kinase promoter.

In other embodiments of the present invention, where secretion of the protein of interest is desired, the vectors are modified by including a signal peptide sequence in operable association with the protein of interest. The sequences of several suitable signal peptides are known to those in the art, including, but not limited to, those derived from tissue plasminogen activator, human growth hormone, lactoferrin, alpha-casein, immunoglobulins and alpha-lactalbumin.

In other embodiments of the present invention, the vectors are modified by incorporating an RNA export element (See, e.g., U.S. Pat. Nos. 5,914,267; 6,136,597; and 5,686,120; and WO99/143 10, all of which are incorporated herein by reference) either 3′ or 5′ to the nucleic acid sequence encoding the protein of interest. It is contemplated that the use of RNA export elements allows high levels of expression of the protein of interest without incorporating splice signals or introns in the nucleic acid sequence encoding the protein of interest.

In still other embodiments, the vector further comprises at least one internal ribosome entry site (IRES) sequence. The sequences of several suitable IRES's are available, including, but not limited to, those derived from foot and mouth disease virus (FDV), encephalomyocarditis virus, and poliovirus. The IRES sequence can be interposed between two transcriptional units (e.g., nucleic acids encoding different proteins of interest or subunits of a multisubunit protein such as an antibody) to form a polycistronic sequence so that the two transcriptional units are transcribed from the same promoter.

The retroviral vectors of the present invention may also further comprise a selectable marker allowing selection of transformed cells. A number of selectable markers find use in the present invention, including, but not limited to the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. In some embodiments, the selectable marker gene is provided as part of polycistronic sequence that also encodes the protein of interest.

Viral vectors, including recombinant retroviral vectors, provide a more efficient means of transferring genes into cells as compared to other techniques such as calcium phosphate-DNA co-precipitation or DEAE-dextran-mediated transfection, electroporation or microinjection of nucleic acids. It is believed that the efficiency of viral transfer is due in part to the fact that the transfer of nucleic acid is a receptor-mediated process (i.e., the virus binds to a specific receptor protein on the surface of the cell to be infected). In addition, the virally transferred nucleic acid once inside a cell integrates in controlled manner in contrast to the integration of nucleic acids which are not virally transferred; nucleic acids transferred by other means such as calcium phosphate-DNA co-precipitation are subject to rearrangement and degradation.

The most commonly used recombinant retroviral vectors are derived from the amphotropic Moloney murine leukemia virus (MOMLV) (See e.g., Miller and Buttimore, Mol. Cell. Biol. 6:2895 [1986]). The MoMLV system has several advantages: 1) this specific retrovirus can infect many different cell types, 2) established packaging cell lines are available for the production of recombinant MoMLV viral particles and 3) the transferred genes are permanently integrated into the target cell chromosome. The established MoMLV vector systems comprise a DNA vector containing a small portion of the retroviral sequence (e.g., the viral long terminal repeat or “LTR” and the packaging or “psi” signal) and a packaging cell line. The gene to be transferred is inserted into the DNA vector. The viral sequences present on the DNA vector provide the signals necessary for the insertion or packaging of the vector RNA into the viral particle and for the expression of the inserted gene. The packaging cell line provides the proteins required for particle assembly (Markowitz et al., J. Virol. 62:1120 [1988]).

The low titer and inefficient infection of certain cell types by MoMLV-based vectors has been overcome by the use of pseudotyped retroviral vectors that contain the G protein of VSV as the membrane associated protein. Unlike retroviral envelope proteins which bind to a specific cell surface protein receptor to gain entry into a cell, the VSV G protein interacts with a phospholipid component of the plasma membrane (Mastromarino et al., J. Gen. Virol. 68:2359 [1977]). Because entry of VSV into a cell is not dependent upon the presence of specific protein receptors, VSV has an extremely broad host range. Pseudotyped retroviral vectors bearing the VSV G protein have an altered host range characteristic of VSV (i.e., they can infect almost all species of vertebrate, invertebrate and insect cells). Importantly, VSV G-pseudotyped retroviral vectors can be concentrated 2000-fold or more by ultracentrifugation without significant loss of infectivity (Bums et al. Proc. Natl. Acad. Sci. USA 90:8033 [1993]).

The present invention is not limited to the use of the VSV G protein when a viral G protein is employed as the heterologous membrane-associated protein within a viral particle (See, e.g., U.S. Pat. No. 5,512,421, which is incorporated herein by reference). The G proteins of viruses in the Vesiculovirus genera other than VSV, such as the Piry and Chandipura viruses, are highly homologous to the VSV G protein and, like the VSV G protein, contain covalently linked palmitic acid (Brun et al. Intervirol. 38:274 [1995] and Masters et al., Virol. 171:285 (1990]). Thus, the G protein of the Piry and Chandipura viruses can be used in place of the VSV G protein for the pseudotyping of viral particles. In addition, the VSV G proteins of viruses within the Lyssa virus genera such as Rabies and Mokola viruses show a high degree of conservation (amino acid sequence as well as functional conservation) with the VSV G proteins. For example, the Mokola virus G protein has been shown to function in a manner similar to the VSV G protein (i.e., to mediate membrane fusion) and therefore may be used in place of the VSV G protein for the pseudotyping of viral particles (Mebatsion et al., J. Virol. 69:1444 [1995]). Viral particles may be pseudotyped using either the Piry, Chandipura or Mokola G protein, with the exception that a plasmid containing sequences encoding either the Piry, Chandipura or Mokola G protein under the transcriptional control of a suitable promoter element (e.g., the CMV intermediate-early promoter; numerous expression vectors containing the CMV IE promoter are available, such as the pcDNA3.1 vectors (Invitrogen)) is used in place of pHCMV-G. Sequences encoding other G proteins derived from other members of the Rhabdoviridae family may be used; sequences encoding numerous rhabdoviral G proteins are available from the GenBank database.

The majority of retroviruses can transfer or integrate a double-stranded linear form of the virus (the provirus) into the genome of the recipient cell only if the recipient cell is cycling (i.e., dividing) at the time of infection. Retroviruses that have been shown to infect dividing cells exclusively, or more efficiently, include MLV, spleen necrosis virus, Rous sarcoma virus and human immunodeficiency virus (HIV; while HIV infects dividing cells more efficiently, HIV can infect non-dividing cells).

It has been shown that the integration of MLV virus DNA depends upon the host cell≦s progression through mitosis and it has been postulated that the dependence upon mitosis reflects a requirement for the breakdown of the nuclear envelope in order for the viral integration complex to gain entry into the nucleus (Roe et al., EMBO J. 12:2099 [1993]). However, as integration does not occur in cells arrested in metaphase, the breakdown of the nuclear envelope alone may not be sufficient to permit viral integration; there may be additional requirements such as the state of condensation of the genomic DNA (Roe et al., supra).

For example, in one such embodiment, the construct backbone comprises one of the constructs described below. In certain of these embodiments, the vector additionally comprises a RNA transport signal (e.g., from woodchuck hepadna virus, WPRE). The Woodchuck hepadna virus post transcriptional enhancer is contemplated to enhance the cytoplasmic levels of RNA and to enhance the translation of the target protein. Tests with retrovector backbone constructs comprising Woodchuck hepadna virus post transcriptional enhancer increase the titer of the MLV-based vectors (presumably by increasing the viral genome transport) and increases the expression of intron-less messages. In particularly preferred embodiments, the Woodchuck hepadna virus post transcriptional enhancer element is inserted in the 3′UTR region of the vector where the remainder of the 3′UTR region is contributed by MLV.

The present invention also contemplates the use of lentiviral vectors to generate high copy number cell lines. The lentiviruses (e.g., equine infectious anemia virus, caprine arthritis-encephalitis virus, human immunodeficiency virus) are a subfamily of retroviruses that are able to integrate into non-dividing cells. The lentiviral genome and the proviral DNA have the three genes found in all retroviruses: gag, pol, and env, which are flanked by two LTR sequences. The gag gene encodes the internal structural proteins (e.g., matrix, capsid, and nucleocapsid proteins); the pol gene encodes the reverse transcriptase, protease, and integrase proteins; and the env gene encodes the viral envelope glycoproteins. The 5′ and 3′ LTRs control transcription and polyadenylation of the viral RNAs. Additional genes in the lentiviral genome include the vif, vpr, tat, rev, vpu, nef, and vpx genes.

A variety of lentiviral vectors and packaging cell lines are known in the art and find use in the present invention (See, e.g., U.S. Pat. Nos. 5,994,136 and 6,013,516, both of which are herein incorporated by reference). Furthermore, the VSV G protein has also been used to pseudotype retroviral vectors based upon the human immunodeficiency virus (HIV) (Naldini et al., Science 272:263 [1996]). Thus, the VSV G protein may be used to generate a variety of pseudotyped retroviral vectors and is not limited to vectors based on MoMLV. The lentiviral vectors may also be modified as described above to contain various regulatory sequences (e.g., signal peptide sequences, RNA export elements, and IRES's). After the lentiviral vectors are produced, they may be used to transfect host cells as described above for retroviral vectors.

A number of exemplary elements are used in the vector constructs are described in Table 1 below. These elements are as follows:

TABLE 1

| | |
|---|---|
| LTR | Retroviral long terminal repeat element containing U3, R, |
| | and U5 |
| Ψ | Psi - retroviral packaging signal (RNA structural element) |
| Kozα | A selectable marker element with a eukaryotic AUG |
| | translation initiation site of the type defined by Kozak |
| | (See e.g., Kozak, Proc. Natl. Acad. Sci. 83: 2850 (1986); |
| | Kozak, Gene 234: 187 [1999]). This could be a |
| | fluorescent protein such as GFP or YFG, a luminescent |
| | protein such as luciferase, or an antibiotic resistance |
| | marker. |
| KozEmpty | No marker present to be expressed from the LTR initiated |
| | mRNA. In some embodiments, to assure lack of ribosome |
| | scanning finding further into the construct and starting |
| | inappropriate translation initiation at the Kb site an |
| | irrelevant (protein) sequence may be inserted at this |
| | location. |
| π | An internal promoter element with a eukaryotic |
| | transcription initiation site to provide for high level |
| | expression and/or cell type-specific expression. |
| SD | Splice donor site optimized for recognition by the |
| | spliceosomal machinery |
| Kozβ | A second selectable marker element with an associated |
| | Kozak-defined translation initiation AUG. This marker |
| | will be expressed only when the ‘capped’ mRNA that is |
| | produced from the promoter π is unspliced as it contains |
| | the first AUG identified by ribosomes during their |
| | ‘scanning’ of the mRNA while seeking the appropriate |
| | codon at which to intiate the translation into the specified |
| | protein. With an -RxRE or RRE- in the construct and |
| | in a cell expressing the Rex or Rev protein splicing |
| | will be inhibited and this marker protein will be expressed. |
| | This marker could be a fluorescent protein such as GFP or |
| | YFG, a luminescent protein such as luciferase, or an |
| | antibiotic resistance marker. |
| SA | A splice acceptor site recognized by the splice machinery |
| | as an indication of the end of an intron |
| -RxRE- | An RNA structural element found within the BLV and |
| | other complex oncoretroviruses to which the Rex protein |
| | attaches and prevents the splicing machinery from |
| | excising the intron from SA . . . SD |
| RRE | An RNA structural element found within the HIV genome |
| | to which the Rev protein attaches and prevents the |
| | splicing machinery from excising the intron from SA . . . SD |
| Koz-GOI | A gene of interest with a translation initiation site AUG |
| | This could be a genomic construct containing introns |
| | where the degree of splicing is variable or unknown. |
| -γ- | An element such as WPRE to assist in nuclear export of |
| | messages |
| -GCE- | Genetic control ‘enhancer’ element to provide a special |
| | type of control to the expression of the gene driven by π. |
| | Enhancers generally operate in a ‘orientation dependent, |
| | position independent manner’ |
| — | Molecular biological connection sites including but not |
| | limited to restriction enzyme sites, recombination sites, or |
| | blunt base-base connections(e.g. Gateway, Cre-LOX) |

Exemplary constructs are described below. The exemplary constructs utilize Rex/RxRe elements. One skilled in the art recognizes that Rev/RRE elements may be substituted accordingly.

The below constructs find use in certain embodiments in the transfection of packaging cells which contain a Rex element in trans relative to this construct. If the vector is to be packaged as MLV, Rex is delivered as a lentiviral vector to ensure a different packaging signal. In some embodiments, the constructs also incorporate a PRE such as WPRE, which will have an additive effect.

(SEQ ID NO:1)

```
  6. LTR-Ψ-Kozα-π-SD-Kozβ-SA-RxRE-LTR
  7. LTR-Ψ-Kozα-π-SD-Kozβ-SA-RxRE-KozGOI-γ-LTR
  8. LTR-Ψ-Kozα-π-SD-Kozβ-RxRE-SA-KozGOI-γ-LTR
  9. LTR-Ψ-Kozα-π-SD-RxRE-Kozβ-SA-KozGOI-γ-LTR
10. LTR-Ψ-Kozα-π-RxRE-SD-Kozβ-SA-KozGOI-γ-LTR
11. LTR-Ψ-Kozα-π-SD-Kozβ-SA-KozGOI-RxRE-γ-LTR
12. LTR-Ψ-Kozα-π-SD-Kozβ-SA-RxRE-KozGOI-LTR
13. LTR-Ψ-Kozα-π-SD-Kozβ-RxRE-SA-KozGOI-LTR
14. LTR-Ψ-Kozα-π-SD-RxRE-Kozβ-SA-KozGOI-LTR
15. LTR-Ψ-Kozα-π-RxRE-SD-Kozβ-SA-KozGOI-LTR
16. LTR-Ψ-Kozα-π-SD-Kozβ-SA-KozGOI-RxRE-LTR
17. LTR-Ψ-Kozα-π-RxRE-SD-GCE-SA-KozGOI-γ-LTR
18. LTR-Ψ-Kozα-π-RxRE-SD-GCE-Kozβ-SA-KozGOI-γ-LTR
19. LTR-Ψ-Kozα-π-RxRE-SD-Kozβ-GCE-SA-KozGOI-γ-LTR
```

Constructs without a selection marker expressed in LTR transcription initiated mRNA

```
20. LTR-Ψ-KozEmpty-π-SD-Kozβ-SA-RxRE-LTR
21. LTR-Ψ-KozEmpty-π-SD-Kozβ-SA-RxRE-KozGOI-γ-LTR
22. LTR-Ψ-KozEmpty-π-SD-Kozβ-RxRE-SA-KozGOI-γ-LTR
23. LTR-Ψ-KozEmpty-π-SD-RxRE-Kozβ-SA-KozGOI-γ-LTR
24. LTR-Ψ-KozEmpty-π-RxRE-SD-Kozβ-SA-KozGOI-γ-LTR
25. LTR-Ψ-KozEmpty-π-SD-Kozβ-SA-KozGOI-RxRE-γ-LTR
26. LTR-Ψ-KozEmpty-π-SD-Kozβ-SA-RxRE-KozGOI-LTR
27. LTR-Ψ-KozEmpty-π-SD-Kozβ-RxRE-SA-KozGOI-LTR
28. LTR-Ψ-KozEmpty-π-SD-RxRE-Kozβ-SA-KozGOI-LTR
29. LTR-Ψ-KozEmpty-π-RxRE-SD-Kozβ-SA-KozGOI-LTR
30. LTR-Ψ-KozEmpty-π-SD-Kozβ-SA-KozGOI-RxRE-LTR
31. LTR-Ψ-KozEmpty-π-RxRE-SD-GCE-SA-KozGOI-γ-LTR
32. LTR-Ψ-KozEmpty-π-RxRE-SD-GCE-Kozβ-SA-KozGOI-
        γ-LTR
33. LTR-Ψ-KozEmpty-π-RxRE-SD-Kozβ-GCE-SA-KozGOI-
        γ-LTR
```

Constructs with no internal promoter where the LTR will control transcription initiation

```
34. LTR-Ψ-KozEmpty-SD-Kozβ-SA-RxRE-LTR
35. LTR-Ψ-KozEmpty-SD-Kozβ-SA-RxRE-KozGOI-γ-LTR
36. LTR-Ψ-KozEmpty-SD-Kozβ-RxRE-SA-KozGOI-γ-LTR
37. LTR-Ψ-KozEmpty-SD-RxRE-Kozβ-SA-KozGOI-γ-LTR
38. LTR-Ψ-KozEmpty-RxRE-SD-Kozβ-SA-KozGOI-γ-LTR
39. LTR-Ψ-KozEmpty-SD-Kozβ-SA-KozGOI-RxRE-γ-LTR
40. LTR-Ψ-KozEmpty-SD-Kozβ-SA-RxRE-KozGOI-LTR
41. LTR-Ψ-KozEmpty-SD-Kozβ-RxRE-SA-KozGOI-LTR
42. LTR-Ψ-KozEmpty-SD-RxRE-Kozβ-SA-KozGOI-LTR
43. LTR-Ψ-KozEmpty-RxRE-SD-Kozβ-SA-KozGOI-LTR
44. LTR-Ψ-KozEmpty-SD-Kozβ-SA-KozGOI-RxRE-LTR
45. LTR-Ψ-KozEmpty-RxRE-SD-GCE-SA-KozGOI-γ-LTR
46. LTR-Ψ-KozEmpty-RxRE-SD-GCE-Kozβ-SA-KozGOI-
        γ-LTR
47. LTR-Ψ-KozEmpty-RxRE-SD-Kozβ-GCE-SA-KozGOI-
        γ-LTR
```

III. Generation of Host Cells Expressing Genes Containing Introns

Following packaging, the retroviral vectors of the present invention are introduced into host cells. Methods for generating host cells using rettoviral vectors are known in the art (See e.g., above description and U.S. Patent Applications Serial Nos. 20040002062 and 20030224415, each of which is herein incorporated by reference in its entirety). A number of mammalian host cell lines are known in the art. In general, these host cells are capable of growth and survival when placed in either monolayer culture or in suspension culture in a medium containing the appropriate nutrients and growth factors, as is described in more detail below. Typically, the cells are capable of expressing and secreting large quantities of a particular protein of interest into the culture medium. Examples of suitable mammalian host cells include, but are not limited to Chinese hamster ovary cells (CHO-K1, ATCC CCl-61); bovine mammary epithelial cells (ATCC CRL 10274; bovine mammary epithelial cells); monkey kidney CV1 line transformed by SV40 (COS-7, ATCC CRL 1651); human embryonic kidney line (293 or 293 cells subcloned for growth in suspension culture; see, e.g., Graham et al., J. Gen Virol., 36:59 [1977]); baby hamster kidney cells (BHK, ATCC CCL 10); mouse sertoli cells (TM4, Mather, Biol. Reprod. 23:243-251 [1980]); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI 38, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 [1982]); MRC 5 cells; FS4 cells; rat fibroblasts (208F cells); MDBK cells (bovine kidney cells); and a human hepatoma line (Hep G2).

In addition to mammalian cell lines, the present invention also contemplates the transfection of plant protoplasts with integrating vectors at a low or high multiplicity of infection. For example, the present invention contemplates a plant cell or whole plant comprising at least one integrated integrating vector, preferably a retroviral vector, and most preferably a pseudotyped retroviral vector. All plants that can be produced by regeneration from protoplasts can also be transfected using the process according to the invention (e.g., cultivated plants of the genera Solanum, Nicotiana, Brassica, Beta, Pisum, Phaseolus, Glycine, Helianthus, Allium, Avena, Hordeum, Oryzae, Setaria, Secale, Sorghum, Triticum, Zea, Musa, Cocos, Cydonia, Pyrus, Malus, Phoenix, Elaeis, Rubus, Fragaria, Prunus, Arachis, Panicum, Saccharum, Coffea, Camellia, Ananas, Vitis or Citrus). In general, protoplasts are produced in accordance with conventional methods (See, e.g., U.S. Pat. Nos. 4,743,548; 4,677,066, 5,149,645; and 5,508,184; all of which are incorporated herein by reference). Plant tissue may be dispersed in an appropriate medium having an appropriate osmotic potential (e.g., 3 to 8 wt. % of a sugar polyol) and one or more polysaccharide hydrolases (e.g., pectinase, cellulase, etc.), and the cell wall degradation allowed to proceed for a sufficient time to provide protoplasts. After filtration the protoplasts may be isolated by centrifugation and may then be resuspended for subsequent treatment or use. Regeneration of protoplasts kept in culture to whole plants is performed by methods known in the art (See, e.g., Evans et al., Handbook of Plant Cell Culture, 1: 124-176, MacMillan Publishing Co., New York [1983]; Binding, Plant Protoplasts, p. 21-37, CRC Press, Boca Raton [1985],) and Potrykus and Shillito, Methods in Enzymology, Vol. 118, Plant Molecular Biology, A. and H. Weissbach eds., Academic Press, Orlando [1986]).

The present invention also contemplates the use of amphibian and insect host cell lines. Examples of suitable insect host cell lines include, but are not limited to, mosquito cell lines (e.g., ATCC CRL-1660). Examples of suitable amphibian host cell lines include, but are not limited to, toad cell lines (e.g., ATCC CCL-102).

The present invention further contemplates the use of stem cell lines. Stem cells may be derived, for example, from embryonic sources (“embryonic stem cells”) or derived from adult sources. For example, U.S. Pat. Nos. 5,843,780 and 6,200,806 to Thompson describes the production of stem cell lines from human embryos. PCT publications WO 00/52145 and WO 01/00650 describe the use of cells from adult humans in a nuclear transfer procedure to produce stem cell lines.

Examples of adult stem cells include, but are not limited to, hematopoietic stem cells, neural stem cells, mesenchymal stem cells, and bone marrow stromal cells. These stem cells have demonstrated the ability to differentiate into a variety of cell types including adipocytes, chondrocytes, osteocytes, myocytes, bone marrow stromal cells, and thymic stroma (mesenchymal stem cells); hepatocytes, vascular cells, and muscle cells (hematopoietic stem cells); myocytes, hepatocytes, and glial cells (bone marrow stromal cells) and, indeed, cells from all three germ layers (adult neural stem cells).

Embryonic stem cells are cells derived from mammalian blastocysts, which are self-renewing and have the ability to yield many or all of the cell types present in a mature animal. Human embryonic stem cell lines suitable for use with the methods and compositions of the present invention include but are not limited to those produced by the following institutions: BresaGen, Inc., Athens, Georgia; CyThera, Inc., San Diego, Calif.; ES Cell International, Melbourne, Australia; Geron Corporation, Menlo Park, Calif.; Goteborg University, Goteborg, Sweden; Karolinska Institute, Stockholm, Sweden; Maria Biotech Co. Ltd.—Maria Infertility Hospital Medical Institute, Seoul, Korea; MizMedi Hospital—Seoul National University, Seoul, Korea; National Centre for Biological Sciences/Tata Institute of Fundamental Research, Bangalore, India; Pochon CHA University, Seoul, Korea; Reliance Life Sciences, Mumbai, India; Technion University, Haifa, Israel; University of California, San Francisco, Calif.; and WiCell Research Institute, Madison, Wis. The human ES cells listed on the Human Embryonic Stem Cell Registry to be created by the National Institutes of Health find use in the methods and compositions of the present invention. However, human ES cells not listed on the NIH registry are also contemplated to find use in embodiments of the present invention (e.g., when it is desirable to prevent ES contamination with nonhuman-derived materials).

The present invention is not limited to the use of human stem cells. Indeed, stem cells from any animal (e.g., bovine) may be utilized in the methods and compositions of the present invention.

The methods and constructs of the present invention are also not limited to the expression of any particular gene or genes of interest. Indeed, the production of a wide variety of proteins is contemplated, including, but not limited to, immunoglobulins, erythropoietin, alpha-interferon, alpha-1 proteinase inhibitor, angiogenin, antithrombin III, beta-acid decarboxylase, human growth hormone, bovine growth hormone, porcine growth hormone, human serum albumin, beta-interferon, calf intestine alkaline phosphatase, cystic fibrosis transmembrane regulator, Factor VIII, Factor IX, Factor X, insulin, lactoferrin, tissue plasminogen activator, myelin basic protein, insulin, proinsulin, prolactin, hepatitis B antigen, immunoglobulin fragments (e.g., FABs), monoclonal antibody CTLA4 Ig, Tag 72 monoclonal antibody, Tag 72 single chain antigen binding protein, protein C, cytokines and their receptors, including, for instance tumor necrosis factors alpha and beta, their receptors and their derivatives; renin; growth hormone releasing factor; parathyroid hormone; thyroid stimulating hormone; lipoproteins; alpha-1 -antitrypsin; follicle stimulating hormone; calcitonin; luteinizing hormone; glucagon; von Willebrands factor; atrial natriuretic factor; lung surfactant; urokinase; bombesin; thrombin; hemopoietic growth factor; enkephalinase; human macrophage inflammatory protein (MIP-1-alpha); a serum albumin such mullerian-inhibiting substance; relaxin A-chain; relaxin B-chain; prorelaxin; mouse gonadotropin-associated peptide; beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-beta; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulinslike growth factor binding proteins; CD proteins such as CD-3, CD-4, CD-8, and CD-19; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressing; regulatory proteins; antibodies; chimeric proteins, such as immunoadhesins, and fragments or fusions of any of the above-listed polypeptides. Nucleic acid and protein sequences for these proteins are available in public databases such as GenBank. Where a particular protein has more than one subunit (such as an immunoglobulin), the genes encoding the sequences may be arranged in polycistronic sequence in the vector, separated by one or more IRES elements. Alternatively, genes encoding different subunits of a protein may be introduced into the host cell on separate vectors. In accordance with the present invention, the gene encoding the protein of interest preferably comprises one or more introns. The introns may be introns normally associated with the gene or may be synthetic or exogenous introns. In some embodiments, the gene may comprise less than its normal complement of introns. For examples, some of the naturally occurring introns may be removed from the gene while others are retained, or one or more of the naturally occurring introns can be replaced by one or more exogenous introns.

III. Production of Transgenic Animals

The present invention contemplates the generation of transgenic animals comprising an exogenous gene of interest comprising introns. In preferred embodiments, the constructs of the present invention are used to create transgenic cell lines and animals, in particular transgenic ungulates, and more particularly transgenic bovine. A variety of methods are known for creating transgenic cell lines and animals.

In some embodiments, the transgenic animal displays an altered phenotype as compared to wild-type animals. Methods for analyzing the presence or absence of such phenotypes include Northern blotting, mRNA protection assays, and RT-PCR. The transgenic animals of the present invention find use as models for testing retroviral therapies, and more generally as systems for research into intron function.

In some embodiments, the transgenic animals made by present invention are used in protein production. It is contemplated that transgenic animals (e.g., bovines) made by the methods and compositions of the present invention may demonstrate increased protein production (See e.g., Palmiter PNAS, 88:478 [1984] and Brinster et al PNAS 85:836 [1988]).

In preferred embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference) and zygote (See e.g., U.S. Patent Application Serial No. 20020129393, which is herein incorporated by reference in its entirety). In other embodiments, the developing non-human embryo can be cultured in vitro to the blastocyst stage. During this time, the blastomeres can be targets for retroviral infection (Jaenisch, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad Sci. USA 82:6927 [1985]). Transfection is easily and efficiently obtained by culturing the blastomeres on a monolayer of virus-producing cells (Van der Putten, supra; Stewart, et al., EMBO J., 6:383 [1987]). Alternatively, infection can be performed at a later stage. Virus or virus-producing cells can be injected into the blastocoel (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involves the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells by retroviral infection and the transduced stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986];

and U.S. Pat. Nos. 6,200,806 and 5,843,780. each of which is herein incorporated by reference in its entirety). Such transduced ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In particularly preferred embodiments, the transgenic animals, and in particular transgenic bovines, are created using a vesicular stomatitis virus (VSV) envelope protein pseudotyped replication defective retroviral gene delivery vector as by the method described in Chan A. W. S., et al., Proc. Natl. Acad. Sci. USA, 95:14028 (1998).

Briefly, most retroviruses only infect dividing cells, because of a critical need for nuclear membrane breakdown to allow the pre-integration complex to contact the chromosomal DNA. The nuclear membrane breakdown that occurs in the oocyte, during metaphase II (MII) of the second meiosis, provides a window during which integration can readily occur. The method described in Chan et al., (gene introduction by injection into the perivitelline space of the acolytes during metaphase II arrest) followed by in vitro fertilization and embryo transfer, provides that nearly 100% of the offspring born will be transgenic heterozygotes.

The approach to transgene insertion described by Chan et al., overcomes four major problems in the more traditional forms of transgenic production currently in use, such as, pronuclear microinjection and nuclear transfer: 1) efficiency of transgenic live births achieved is a hundred-fold higher that of other methods; 2) genes insert as single copies, with less risk of genetic instability upon subsequent cell replication; 3) transgenes are inserted prior to fertilization, eliminating mosaicism; and 4) animals (i.e., bovine calves) undergo normal gestation and birth. Evaluation of second generation transgenic animals (i.e., bovine) produced by the Chan et al., method show Mendelian inheritance and gene stability.

IV. Gene Therapy Using Intron Containing Genes of Interest

The present invention also provides methods and compositions suitable for gene therapy to deliver a gene of interest with introns intact. The methods described below are generally applicable across many species susceptible to infection by complex retroviruses.

Viral vectors commonly used for in vivo or ex vivo targeting and gene therapy procedures are DNA-based vectors and retroviral vectors. In preferred embodiments, genes are introduced in a retroviral vector (e.g., as described in U.S. Pat. Nos. 6,794,188, 5,399,346, 4,650,764, 4,980,289 and 5,124,263; all of which are herein incorporated by reference; Mann et al., Cell, 33:153 [1983]; Markowitz et al., J. Virol., 62:1120 [1988]; PCT/US95/14575; EP 453242; EP178220; Bernstein et al., Genet. Eng., 7:235 [1985]; McCormick, BioTechnol., 3:689 [1985]; WO 95/07358; and Kuo et al., Blood, 82:845 [1993]). The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“pleen necrosis virus”); RSV (“Rous sarcoma virus”) and Friend virus. Defective retroviral vectors are also disclosed in WO95/02697.

In general, in order to construct recombinant retroviruses containing a nucleic acid sequence, a plasmid is constructed that contains the LTRs, the encapsidation sequence and the coding sequence. This construct is used to transfect a packaging cell line, which cell line is able to supply in trans the retroviral functions that are deficient in the plasmid. In general, the packaging cell lines are thus able to express the gag, pol and env genes. Such packaging cell lines have been described in the prior art, in particular the cell line PA317 (U.S. Pat. No.: 4,861,719, herein incorporated by reference), the PsiCRIP cell line (See, WO90/02806), and the GP+envAm-12 cell line (See, WO89/07150). In addition, the recombinant retroviral vectors can contain modifications within the LTRs for suppressing transcriptional activity as well as extensive encapsidation sequences that may include a part of the gag gene (Bender et al., J. Virol., 61:1639 [1987]). Recombinant retroviral vectors are purified by standard techniques known to those having ordinary skill in the art.

The retroviral vector carrying the nucleic acid sequence of interest may be administered to an individual in need of such therapy in a variety of ways. Retroviral supernatants of host cells transduced with retroviruses of the present invention and producing the virus may be administered to the individual in need of gene therapy. Additionally, a substantially purified form of the virus may be administered to the mammal in need of such treatment alone or in the form of a pharmaceutical composition.

Alternatively, the gene therapy may be accomplished by inserting the nucleic acid sequences encoding the therapeutic protein(s) into the recombinant retrovirus vector and introducing it into a host cell. The host cell, which contains the recombinant retroviral vector and expresses the desired therapeutic protein retaining introns is then administered to or implanted in the individual in need of gene therapy. The cells then express the therapeutic protein recombinantly in the mammal.

Means of administering the host cell containing the recombinant retroviral vectors of the invention that recombinantly express the proteins of interest include, but are not limited to, intravenous, intramuscular, intralesional, subcutaneous or intraperitoneal injection or implantation. Alternatively, the cells containing the recombinant retroviral vectors may be administered locally by topical application, direct injection into an affected area or implantation of a porous device containing cells from the host or another species in which the recombinant retroviral vectors are inserted and which express the proteins of interest.

Examples of diseases that may be suitable for gene therapy include, but are not limited to, neurodegenerative diseases or disorders, Alzheimer's, schizophrenia, epilepsy, neoplasms, cancer and AIDS or other diseases requiring replacement or the up or down regulation of a gene of interest.

IV. Research and Diagnostic Applications

The present invention further provides for the use of retroviral vectors for the expression of genes of interest comprising introns in host cells. Such host cells find use in a variety of research applications. For example, in some embodiments, host cells are transduced with vectors with/without introns and the differences in gene function are compared.

In a further embodiment the RxRe luciferase reporter system described in the experimental section below is used as a diagnostic test to identify the presence of Rex in cells and hence to show prior infection with BLV. For example, in some embodiments, B cells from a cow are collected and transduced with the vectors described herein (e.g., the luciferase reporter vector described in Example 1). If Rex is present in the cell, the luciferase is expressed. In other embodiments, a construct containing an RRE-luciferase element is used as an HIV diagnostic.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); and C (degrees Centigrade).

### Example 1

### Development and Testing of a Bovine Leukemia Virus (BLV) RNA Response Element Reporter System for Evaluation of Transdominant Rex Mutants

This example describes an experiment designed to determine if BLV (bovine leukemia virus) infected cells that carry a transdominant negative Rex mutant (TD-Rex) inhibit BLV replication.

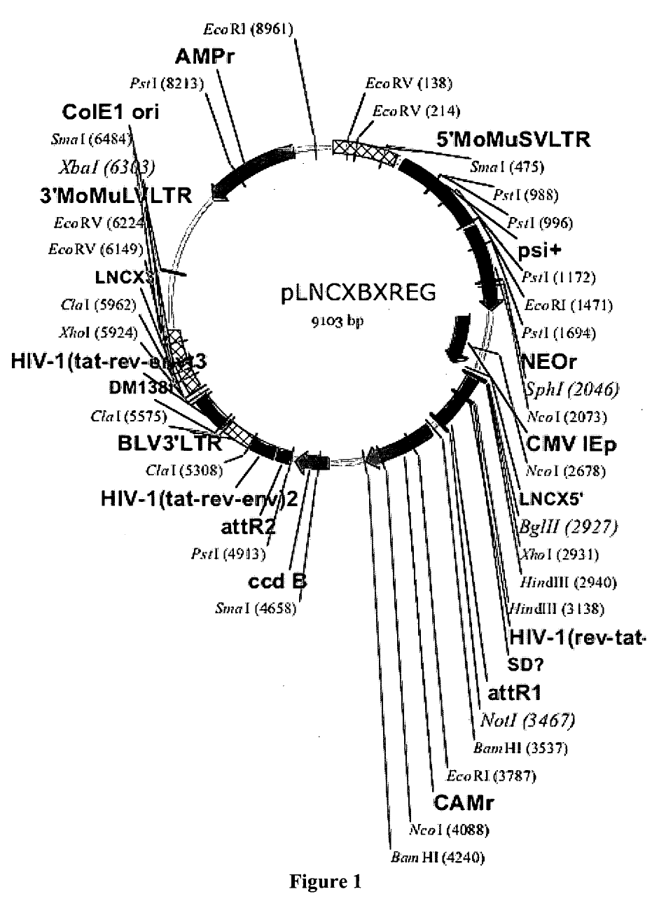

Splice sites and BLV RexRE sequences were derived from pDM138 and subcloned into the retroviral vector pLNCX2 (Clontech) along with a GATEWAY reading frame cassette (RfA; Invitrogen) to create the GATEWAY destination retrovector, pLNCXBXREG. This retrovector was recombined with the Luciferase entry clone pENTR1A/Luc to derive the RexRE reporter vector pLNCXBXRE/Luc (FIG. 1; SEQ ID NO:1).

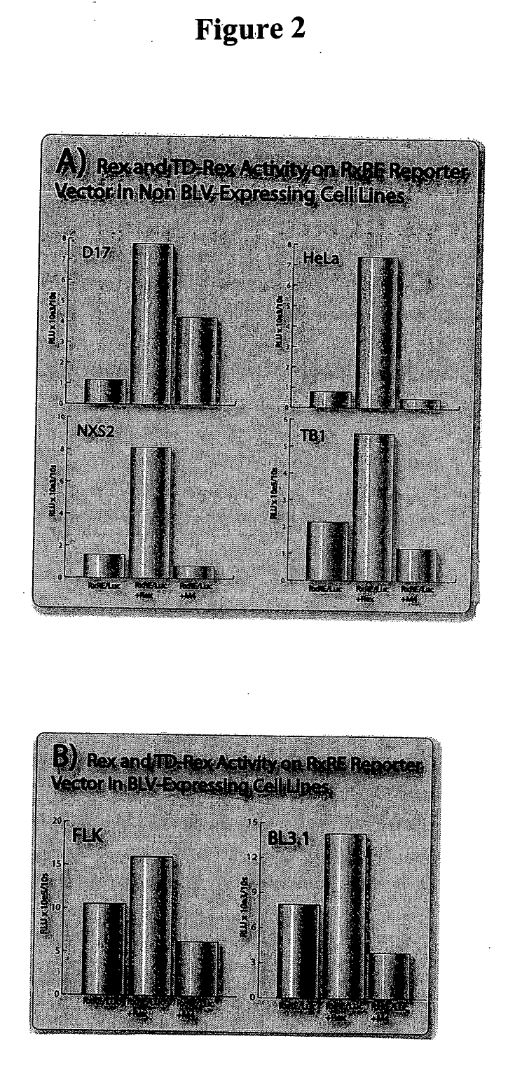

Non-BLV expressing cell lines D17, HeLa, NXS2, and TB1 or BLV expressing cell lines BL3.1 and FLK were transduced with the retroviral vector LNCXBXRE/Luc (RexRE/Luc). These cells were subsequently transduced with retrovectors expressing wild type BLV Rex (RexRE/Luc+Rex) or the TD-Rex mutant M4 (RexRE/luc+M4). Luciferase expression was measured on 5×10⁴ cells and recorded as Relative Light Units (RLU) over a 10 s interval. Mean and standard deviation were derived from data of at least 10 experiments. The results are shown in FIG. 2. The results indicate that high levels of luciferase are expressed in cells expressing BLV or a wild type Rex trans-gene.

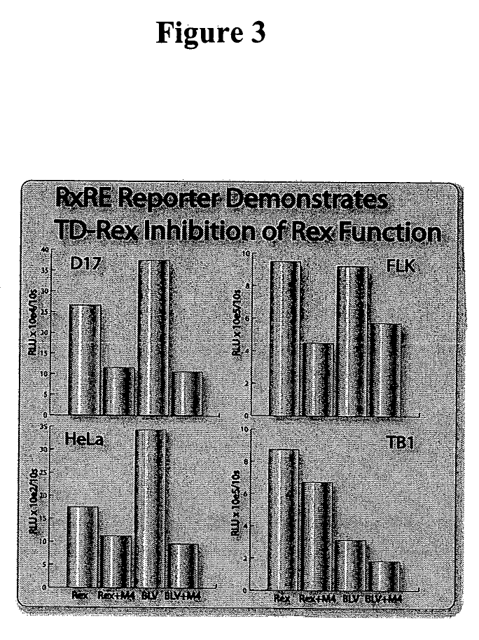

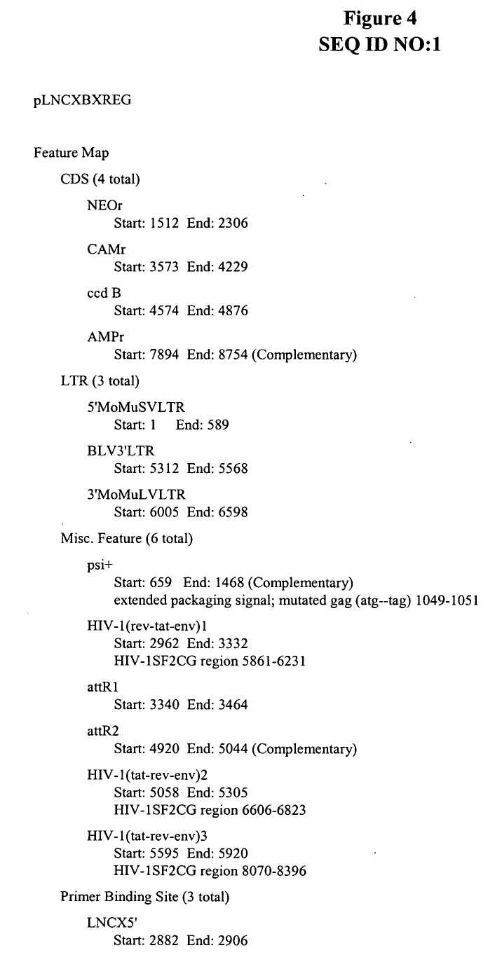

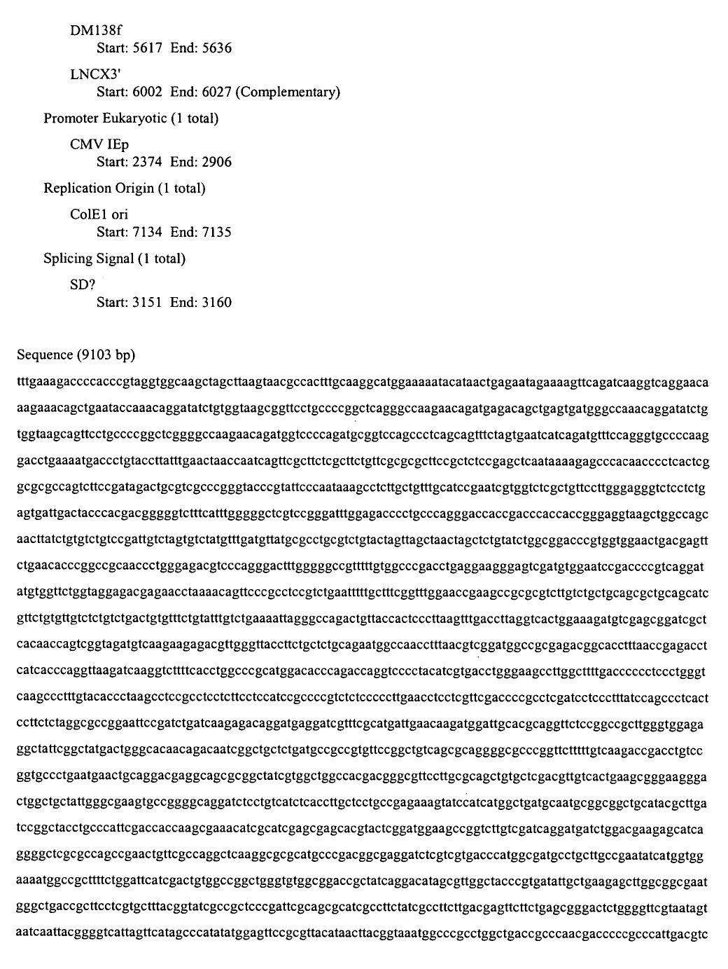

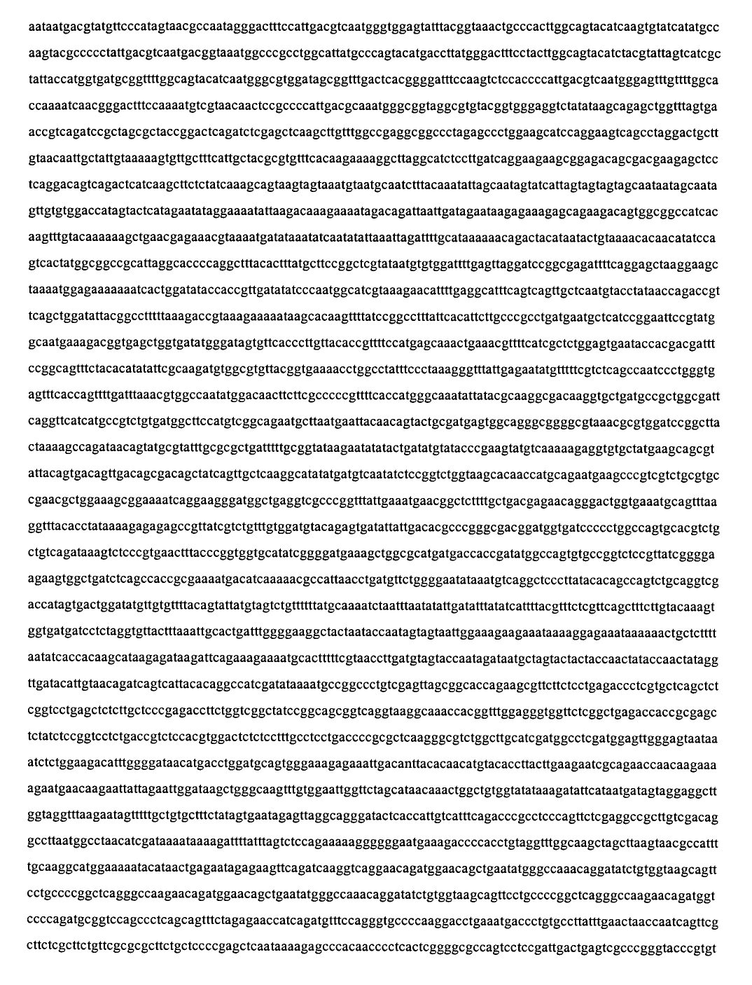

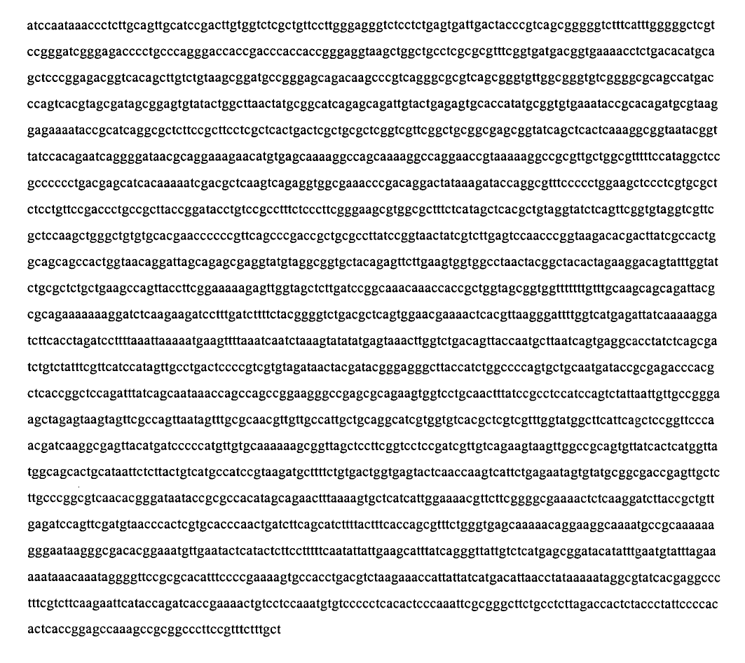

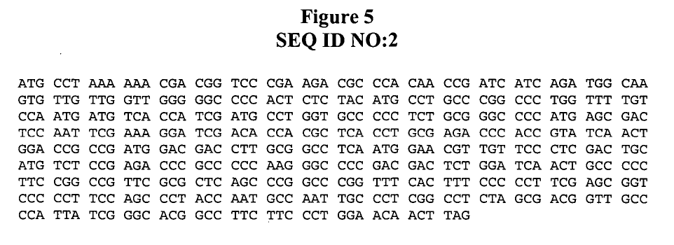

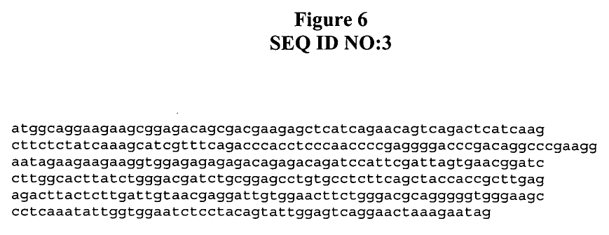

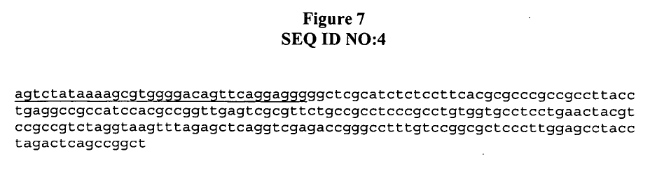

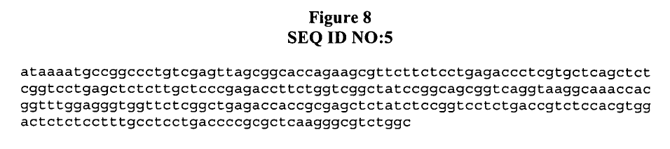

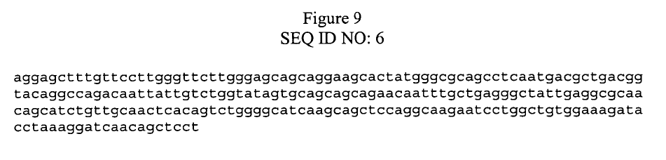

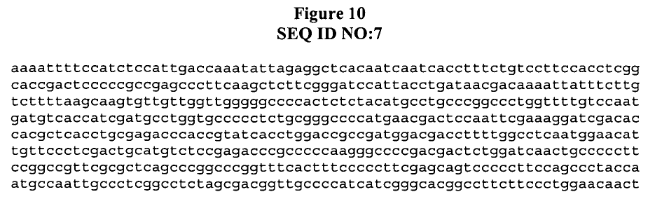

The ability of the RexRE reporter vector to demonstrate mutant trans dominant-Rex inhibition of BLV provirus-induced activity was also investigated. Retroviral vector LNCXBXRE/Luc (RexRE/Luc) transduced cell lines D17, HeLa, NXS2, and TB1 were transiently transfected with plasmid expressing YFP (Cntrl) or co-transfected with plasmids expressing the BLV provirus, pBLV913, and either YFP (BLV), TD-Rex mutant M4-YFP (BLV+M4), or wild-type Rex-YFP (BLV+Rex). Luciferase expression was measured on 5×103 cells and recorded as Relative Light Units (RLU) over a 10 s interval. Mean and standard deviation were derived from data of three experiments. Results are shown in FIG. 3. The results indicate that expression of TD-Rex transgene reduces luciferase levels.

In conclusion, this example demonstrates that BLV or Rex expression significantly increased luciferase expression, while TD-Rex significantly decreased Luciferase expression.

The luciferase-RxRe reporter construct was designed with splice sites flanking the luciferase gene. The expectation would be that luciferase would not be expressed from this construct because of splicing occurring as the gene is transcribed and nuclear export occurs. In the presence of Rex or wild type Rex from BLV and RxRe, splicing did not occur, demonstrating the ability of Rex to protect transgene RNA from splicing during nuclear export. When TD Rex was present and bound to Rex the effect was diminished.

### Example 2

### Effect of Brex on Packaging of Intron-Containing Elements

This example describes the effect of Brex during retroviral packaging on packaging of intron-containing sequence elements located between the two LTRs.

A. Materials and Methods

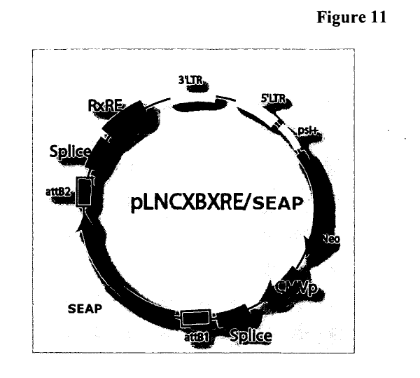

Human secreted endogenous alkaline phosphatase (SEAP, Gene Therapy Systems, San Diege, Calif.) was introduced into the existing pLNCXBXRE vector (FIG. 11). This construct is based on the reporter gene construct pDM138 (Popa et al., Mol Cell Biol 2002; 22:2057-67) where the luciferase reporter gene is flanked by a splice donor and a splice acceptor site. In addition the construct contains the BLV derived RxRE which upon binding to the Rex protein induces nuclear export of the transcript (Choi and Hope, J. Virol. 2005, 79:7172-7181).

Endotoxin-free preparations of pLNCXBXRE/SEAP (SEQ ID NO:8), pBrex, and pVSV-G (used for pseudotyping the retroviral particles) were made and the following ratios of each plasmid were used to perform transient transfection of 90% confluent 293GP cells in 6-well plates using the Lipofectamine 2000 reagent from Invitrogen (San Diego, Calif.):

TABLE 2
Different ratios of each plasmid were used in a 4 microgram/reaction
using the lipofectamine 2000 reagent from Invitrogen (San Diego, CA).

| | μg | | | | | |
|---|---|---|---|---|---|---|
| | 1:1 | 2:1 | 4:1 | 8:1 | 16:1 | 32:1 |
| pVSVG | 1.33 | 1.33 | 1.33 | 1.33 | 1.33 | 1.33 |
| pLNCXBXRE-SEAP | 1.33 | 1.33 | 1.33 | 1.33 | 1.33 | 1.33 |
| pBRex | 1.33 | 0.67 | 0.33 | 0.17 | 0.08 | 0.04 |
| pDrive | 0.00 | 0.67 | 1.00 | 1.17 | 1.25 | 1.29 |
| | 4 | 4 | 4 | 4 | 4 | 4 |

Lipofectamine and DNA were mixed to form complexes and after 20 min added to confluent 293GP packaging cells. 48 hours later, supernatant containing infectious particles was harvested, filtered through 0.45 micron filter and added to 2.5×10⁵ CHO host cells in the presence of 8 microgram/ml of polybrene. One day after transduction, selection using G418 (Hyclone, Logan, UT) was initiated and after 10 days, supernatant from each of the 6 pools was analyzed for the presence of SEAP. The SEAP assay includes collecting supernatant, heat-inactivating the sample for 30 min at 65° C. and then developing using the PNPP reagent (Pierce, Rockford, Ill.). This reagent induces a color reaction depending on the concentration of alkaline phosphatase. Quantification is done in a Microtiter plate reader at 405 nm wavelength using kinetic settings.

B. Results

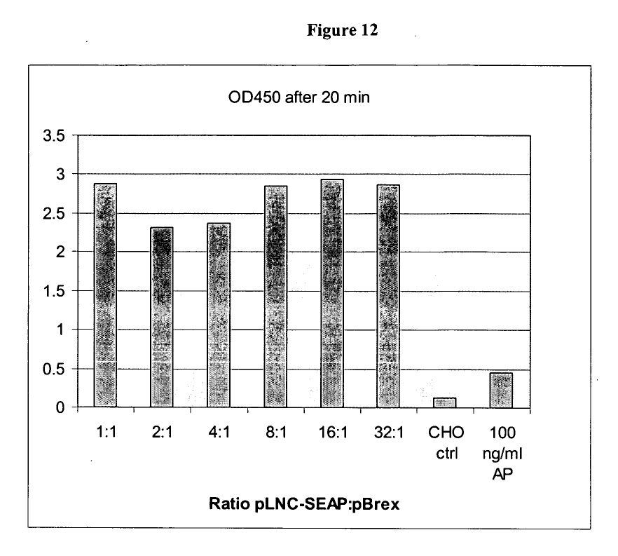

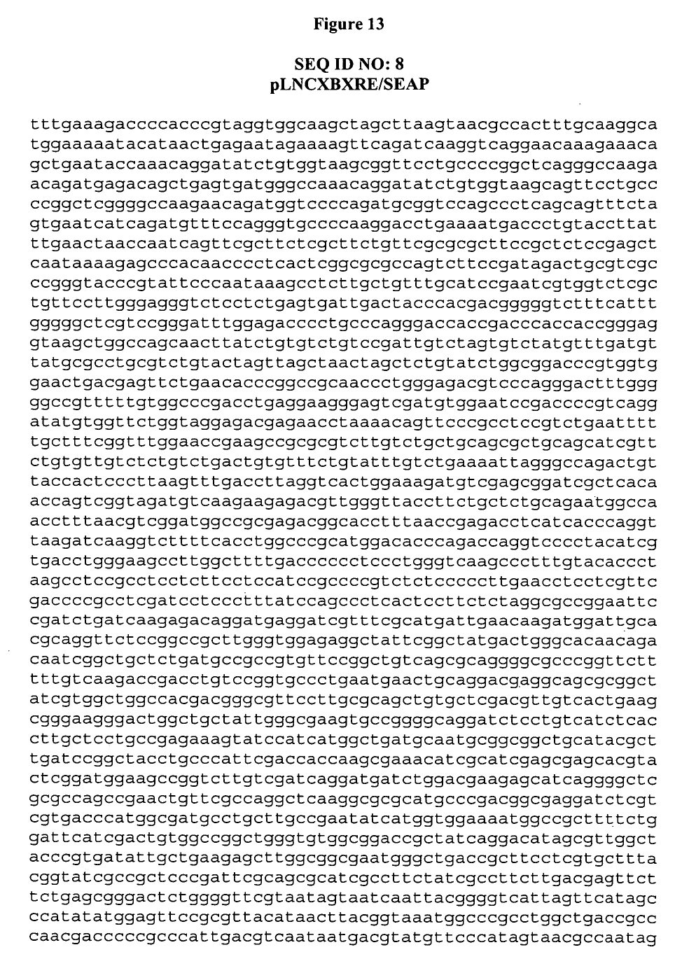

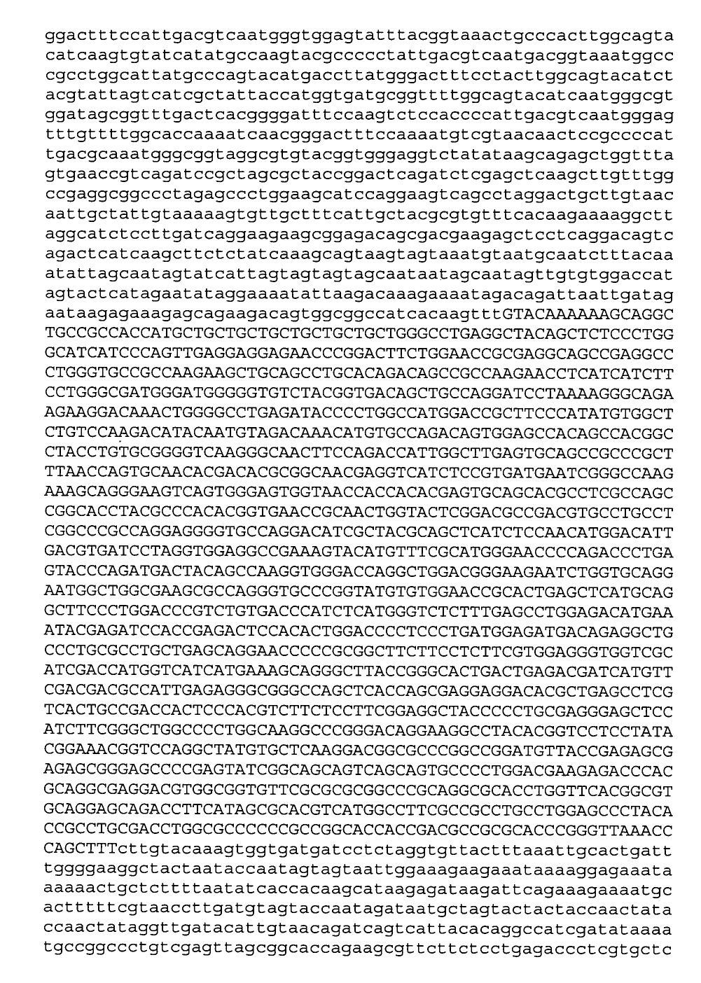

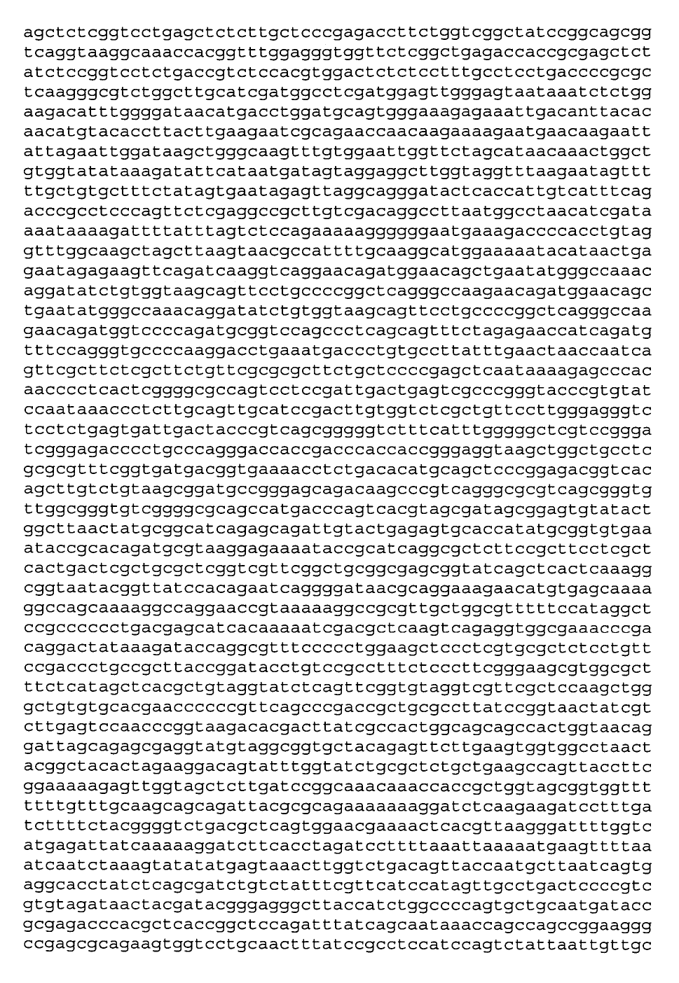

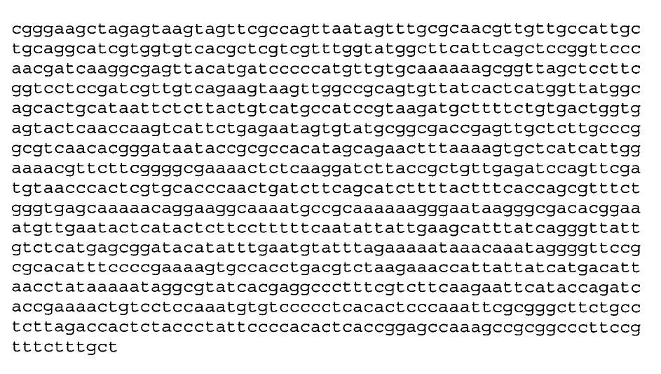

Earlier data obtained with the luciferase reporter gene (See e.g., Example 1) demonstrated that in the absence of the Brex protein, only background level of luciferase activity was detectable, indicating that the HIV-derived splice sites that flank the luciferase gene are effectively splicing out the luciferase gene. By performing the transfection in the presence of various amounts of the Brex protein (regulated through varying amounts of pBREX added to the transfection mix as shown in Table 2), the role of the Brex protein during retroviral packaging was tested. Results are shown in FIG. 12. When CHO host cell pools that were transduced with supernatants of the 6 transfection events are analyzed, the presence of pBREX during packaging helped prevent splicing of the SEAP gene as is evident by the strong SEAP expression seen in all the transduced pools even at the 32:1 ratio of pLNCXBXRE-SEAP vs pBREX. The data demonstrate that the activity of Brex using the luciferase reporter gene is not dependent on the gene that is flanked by that splice sites, rather that it is a highly efficient mechanism to prevent splicing of all genes as long as the Brex protein and Rex-responsive element is present during packaging.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A system, comprising:
a) a retroviral vector comprising a promoter operably linked to a nucleic acid encoding an exogenous gene and a nucleic acid encoding an RNA export protein response element; and
b) a packaging cell line expressing an RNA export protein.

2. The system of claim 1, wherein said RNA export protein response element is a Rex RNA response element (RxRE).

3. The system of claim 2, wherein said RxRE is selected from the group consisting of a bovine leukemia virus RxRE and a human T-cell leukemia RxRe.

4. The system of claim 3, wherein said bovine leukemia virus RxRE is at least 90% identical to SEQ ID NO:5.

5. The system of claim 3, wherein said bovine leukemia virus RxRE has the nucleic acid sequence of SEQ ID NO:5.

6. The system of claim 3, wherein said human T Cell leukemia virus RxRE is at least 90% identical to SEQ ID NO:4.

7. The system of claim 3, wherein said human T Cell leukemia virus RxRE has the nucleic acid sequence of SEQ ID NO:4.

8. The system of claim 1, wherein said RNA export protein response element is a human immunodeficiency virus RRE.

9. The system of claim 8, wherein said human immunodeficiency virus RRE is at least 90% identical to SEQ ID NO: 6.

10. The system of claim 8, wherein said human immunodeficiency virus RRE has the nucleic acid sequence of SEQ ID NO: 6.

11. The system of claim 1, wherein said RNA export protein is selected from the group consisting of a bovine leukemia virus Rex and a human T-cell leukemia virus Rex.

12. The system of claim 11, wherein said bovine leukemia virus Rex is at least 90% identical to SEQ ID NO:2.

13. The system of claim 11, wherein said bovine leukemia virus Rex has the nucleic acid sequence of SEQ ID NO:2.

14. The system of claim 11, wherein said human T-cell leukemia virus Rex is at least 90% identical to SEQ ID NO:7.

15. The system of claim 11, wherein said human T-cell leukemia virus Rex has the nucleic acid sequence of SEQ ID NO:7.

16. The system of claim 1, wherein said nuclear export protein is human immunodeficiency virus Rev.

17. The system of claim 16, wherein said human immunodeficiency virus Rev is at least 90% identical to SEQ ID NO:3.

18. The system of claim 16, wherein said human immunodeficiency virus Rev has the nucleic acid sequence of SEQ ID NO:3.

19. The system of claim 1, wherein said RNA export protein is present on a second vector.

20. The system of claim 19, wherein said second vector is a lentiviral vector or MLV vector.

21. The system of claim 19, wherein said second vector is an inducible expression vector.

22. The system of claim 1, wherein said RNA export protein is present as a transgene.

23. A method, comprising:
a) providing
i) a retroviral vector comprising a promoter operably linked to a nucleic acid encoding an exogenous gene and a nucleic acid encoding an RNA export protein response element; and
ii) a packaging cell line expressing an RNA export protein; and
b) introducing said retroviral vector into said packaging cell line under conditions such that said retroviral vector is packaged without introns being spliced from said exogenous gene.

24. A retroviral vector comprising a promoter operably linked to a nucleic acid encoding an exogenous gene and a nucleic acid encoding an RNA export protein response element.

25. The retroviral vector of claim 24, wherein said retroviral vector further comprises an RNA stabilizing element.

26. The retroviral vector of claim 25, wherein said RNA stabilizing element is a WPRE.

27. A method, comprising
a) providing
i) a cell suspected of harboring a viral infection; and
ii) a retroviral vector comprising a promoter operably linked to a nucleic acid encoding an exogenous gene and a nucleic acid encoding an RNA export protein response element, wherein said retroviral vector further comprises a reporter gene; and
b) transfecting said cell with said retroviral vector under conditions such that said reporter gene is expressed in the presence but not in the absence of said viral infection.


SEQUENCE LISTING (7 sequences)

SEQ ID NO: 1 - DNA, 9103 bp, Artificial Sequence
  Synthetic
tttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattccgatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttgtttggccgaggcggccctagagccctggaagcatccaggaagtcagcctaggactgcttgtaacaattgctattgtaaaaagtgttgctttcattgctacgcgtgtttcacaagaaaaggcttaggcatctccttgatcaggaagaagcggagacagcgacgaagagctcctcaggacagtcagactcatcaagcttctctatcaaagcagtaagtagtaaatgtaatgcaatctttacaaatattagcaatagtatcattagtagtagtagcaataatagcaatagttgtgtggaccatagtactcatagaatataggaaaatattaagacaaagaaaatagacagattaattgatagaataagagaaagagcagaagacagtggcggccatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatgatcctctaggtgttactttaaattgcactgatttggggaaggctactaataccaatagtagtaattggaaagaagaaataaaaggagaaataaaaaactgctcttttaatatcaccacaagcataagagataagattcagaaagaaaatgcactttttcgtaaccttgatgtagtaccaatagataatgctagtactactaccaactataccaactataggttgatacattgtaacagatcagtcattacacaggccatcgatataaaatgccggccctgtcgagttagcggcaccagaagcgttcttctcctgagaccctcgtgctcagctctcggtcctgagctctcttgctcccgagaccttctggtcggctatccggcagcggtcaggtaaggcaaaccacggtttggagggtggttctcggctgagaccaccgcgagctctatctccggtcctctgaccgtctccacgtggactctctcctttgcctcctgaccccgcgctcaagggcgtctggcttgcatcgatggcctcgatggagttgggagtaataaatctctggaagacatttggggataacatgacctggatgcagtgggaaagagaaattgacanttacacaacatgtacaccttacttgaagaatcgcagaaccaacaagaaaagaatgaacaagaattattagaattggataagctgggcaagtttgtggaattggttctagcataacaaactggctgtggtatataaagatattcataatgatagtaggaggcttggtaggtttaagaatagtttttgctgtgctttctatagtgaatagagttaggcagggatactcaccattgtcatttcagacccgcctcccagttctcgaggccgcttgtcgacaggccttaatggcctaacatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcataccagatcaccgaaaactgtcctccaaatgtgtccccctcacactcccaaattcgcgggcttctgcctcttagaccactctaccctattccccacactcaccggagccaaagccgcggcccttccgtttctttgct

SEQ ID NO: 2 - DNA, 471 bp, Bovine leukemia virus
atgcctaaaaaacgacggtcccgaagacgcccacaaccgatcatcagatggcaagtgttgttggttgggggccccactctctacatgcctgcccggccctggttttgtccaatgatgtcaccatcgatgcctggtgccccctctgcgggccccatgagcgactccaattcgaaaggatcgacaccacgctcacctgcgagacccaccgtatcaactggaccgccgatggacgaccttgcggcctcaatggaacgttgttccctcgactgcatgtctccgagacccgcccccaagggccccgacgactctggatcaactgcccccttccggccgttcgcgctcagcccggcccggtttcactttcccccttcgagcggtcccccttccagccctaccaatgccaattgccctcggcctctagcgacggttgccccattatcgggcacggccttcttccctggaacaacttag

SEQ ID NO: 3 - DNA, 360 bp, Human immunodeficiency virus
atggcaggaagaagcggagacagcgacgaagagctcatcagaacagtcagactcatcaagcttctctatcaaagcatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatag

SEQ ID NO: 4 - DNA, 225 bp, Human T-cell leukemia virus
agtctataaaagcgtggggacagttcaggagggggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggct

SEQ ID NO: 5 - DNA, 257 bp, Bovine leukemia virus
ataaaatgccggccctgtcgagttagcggcaccagaagcgttcttctcctgagaccctcgtgctcagctctcggtcctgagctctcttgctcccgagaccttctggtcggctatccggcagcggtcaggtaaggcaaaccacggtttggagggtggttctcggctgagaccaccgcgagctctatctccggtcctctgaccgtctccacgtggactctctcctttgcctcctgaccccgcgctcaagggcgtctggc

SEQ ID NO: 6 - DNA, 234 bp, Human immunodeficiency virus
aggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 7 - DNA, 568 bp, Human T-cell leukemia virus
aaaattttccatctccattgaccaaatattagaggctcacaatcaatcacctttctgtccttccacctcggcaccgactcccccgccgagcccttcaagctcttcgggatccattacctgataacgacaaaattatttcttgtcttttaagcaagtgttgttggttgggggccccactctctacatgcctgcccggccctggttttgtccaatgatgtcaccatcgatgcctggtgccccctctgcgggccccatgaacgactccaattcgaaaggatcgacaccacgctcacctgcgagacccaccgtatcacctggaccgccgatggacgaccttttggcctcaatggaacattgttccctcgactgcatgtctccgagacccgcccccaagggccccgacgactctggatcaactgcccccttccggccgttcgcgctcagcccggcccggtttcactttcccccttcgagcagtcccccttccagccctaccaatgccaattgccctcggcctctagcgacggttgccccatcatcgggcacggccttcttccctggaacaact